(19) United States
(12) Patent Application Publication
(10) Pub. No.: US 2026/0174634 A1
(43) Pub. Date: Jun. 25, 2026

(54) CONTINUOUS MID-AIR 3-DIMENSIONAL PRINTING FOR PHARMACEUTICAL DOSAGE FORMS

(71) Applicant: Board of Regents, The University of Texas System, Austin, TX (US)

(72) Inventors: Leela Raghava Jaidev Chakka, Austin, TX (US); Mohammed Maniruzzaman, Austin, TX (US); Vineet R. Kulkarni, Austin, TX (US); Faez Alkadi, Austin, TX (US)

(21) Appl. No.: 19/124,468

(22) PCT Filed: Oct. 27, 2023
(86) PCT No.: PCT/US2023/078093

§ 371 Date: Apr. 25, 2025

Related U.S. Application Data
(60) Provisional application No. 63/420,446, filed on Oct. 28, 2022.

(51) Int. Cl. B33Y 30/00

(52) U.S. Cl. CPC A61J 3/007; A61K 9/2031; A61K 9/2054; A61K 9/2086; A61K 9/28; A61K 31/216; A61K 31/4422; B29C 64/118; B29C 64/386; B33Y 10/00; B33Y 50/00; B33Y 70/10; B33Y 80/00; B29K 2001/08; B29K 2071/02; B29K 2105/0035

(57) ABSTRACT

Described are continuous manufacturing methods using 3D printing or any similar additive manufacturing technology to produce pharmaceutical dosage forms or pharmaceutical delivery devices. Manufacturing methods using hot-melt extrusion to fabricate filaments for fused deposition modeling (FDM) based 3D printing are disclosed. Methods using FDM based 3D printing to fabricate printed products suitable for pharmaceutical delivery purposes are disclosed. The printed products may have various shapes and configurations.

### CROSS-REFERENCE TO RELATED APPLICATIONS

This application claims the benefit of and priority to U.S. Provisional Application No. 63/420,446, filed on Oct. 28, 2022, which is hereby incorporated by reference in its entirety.

### FIELD

The present disclosure relates generally to additive manufacturing technology, and more specifically to the production of various pharmaceutical dosage forms of using continuous extrusion and mid-air 3D printing systems.

### BACKGROUND

Personalized medication and personalized pharmaceutical dosing is a current and promising field of research. Many 3D printing technologies utilize additive deposition of melted feedstock (e.g., filament) extruded through a computer-controlled deposition nozzle to build product with desired geometry and structure.

### SUMMARY

As polymeric materials are widely-used as 3D printing feedstocks and hot-melt extrusion is a high-efficiency industrial process of manipulating polymeric materials, a continuous manufacturing technology that connects manufacturing feedstock by hot-melt extrusion and 3D printing can achieve mass production of various pharmaceutical dosage forms with precision control over the active pharmaceutical ingredient (API) loading throughout such forms.

The present disclosure relates generally to personalized medication and pharmaceutical dosage fabricated by hot-melt extrusion and 3D printing technologies. As described herein, embodiments described herein have been applied to methods and systems for fabricating different types of medication, pharmaceutical dosage, or pharmaceutical delivery carriers with various configurations.

In an aspect, methods of manufacturing printed products are provided. An example method comprises providing an initial mixture including at least one polymeric material and at least one active pharmaceutical ingredient (API), feeding the initial mixture into a heating barrel, heating the initial mixture to a temperature that reduces the viscosity of the at least one polymeric material, pumping the initial mixture through a die to form a filament, heating the filament to a fusing temperature, and depositing the filament on a receiving surface at an oblique angle to form a printed product. In some embodiments, the polymeric material comprises a thermoplastic material or component, such as a digestible or biocompatible thermoplastic. In some examples, the filament contains the at least one API in crystalline state, semi-crystalline state, or amorphous state. In some examples, the receiving surface is a dynamic bed that moves continuously along an axis and wherein the method further comprises transporting the printed product on the dynamic bed. In some examples, the dynamic bed is a conveyor belt. In some examples, the method further comprises measuring a quality or characteristic of the printed product using at least one in-line measurement device. In some examples, the at least one in-line measurement device includes a digital imaging unit. In some examples, the at least one measurement device includes an in-line UV-VIS imaging unit. Optionally, the at least one measurement device includes an infrared spectrometer, a back pressure sensor, and a NIR probe.

In some examples, the method further comprises injecting a second API into the printed product using a syringe. In some examples, the second API is a gel or has a lipid-based formulation. In some examples, the syringe is a mechanical or pressure assisted syringe. In some examples, the initial mixture is fed into the heating barrel via volumetric feeding. In some examples, the printed product is in the form of a tablet, a pill, a thin film, an orally dissolvable film, a transdermal patch, or microneedles. In some examples, the initial mixture further comprises one or more excipients.

In another aspect, methods of manufacturing filaments are provided. An example method of this aspect comprises providing an initial mixture including at least one polymeric material and at least one active pharmaceutical ingredient (API), feeding the initial mixture into a heating barrel, heating the initial mixture at a temperature that reduces the viscosity of the at least one polymeric material, and pumping the initial mixture through a die to form a filament. In some examples, the initial mixture includes a thermoplastic, such as a biocompatible or digestible thermoplastic. In some examples, the filament contains at least one API in semi-crystalline state. In some examples, the filament contains at least one API in crystalline state.

In another aspect, printed products are provided. An example printed product comprises at least one active pharmaceutical ingredient (API) and at least one polymeric material, wherein the printed product comprises a fused multilayer structure with one or more layers of the fused multilayer structure comprising the at least one active pharmaceutical ingredient (API) and the at least one polymeric material. In examples, the polymeric material comprises a biocompatible or digestible thermoplastic. Optionally, layers of the fused multilayer structure are arranged at an oblique angle to a surface of the printed product. In some examples, the API is in a gel state or a liquid state. In some examples, the API is in a crystalline state. In some examples, the API is in a semi-crystalline state or an amorphous state. In some examples, the at least one API is Nifedipine, Aspirin, chloroquine diphosphate, or Ibuprofen. In some examples, the at least one polymeric material is hydroxpropylmethyl cellulose, hydroxypropyl cellulose, or hydroxpropylmethyl cellulose. In some examples, the printed product further comprises at least one plasticizer or at least one excipient. Optionally, the at least one plasticizer is polyethylene oxide or Soluplus.

In some examples, the printed product further comprises a coating surrounding at least a portion of the fused multilayer structure. In some examples, the coating comprises a second fused multilayer structure surrounding at least a portion of the fused multilayer structure. In some examples, the at least one API or the at least one polymeric material has a porosity greater than zero. In some examples, the printed product further comprises a semi-solid substance that is at least partially surrounded by or at least partially internal to the fused multilayer structure. In some examples, the semi-solid substance is a gel or has a lipid-based formulation. In some examples, the fused multilayer structure comprises a first set of fused layers including a first API and a second set of fused layers including a second API, wherein the first set of fused layers and the second set of fused layers are fused to one another. In some examples, the fused multilayer structure comprises a core part and a shell part, wherein the core part includes a first set of fused layers including at least one API, wherein the shell part includes fused material surrounding the core part. Optionally, the core part and the shell part have different densities. In some examples, the printed product further comprises a markline in the fused multilayer structure, wherein the markline comprises material deposited on an outer surface of the fused multilayer structure or a recessed region in the fused multilayer structure.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 is a schematic illustration of manufacturing filaments according to some examples.

FIG. 2A is a block diagram of manufacturing printed products according to some examples.

FIG. 2B is a flowchart providing an overview of a method of manufacturing printed products according to some examples.

FIG. 2C is an example fused deposition modeling (FDM)-based 3D-printing system including a dynamic platform according to some examples.

FIG. 2D is an example conventional FDM-based 3D-printing system according to some examples.

FIG. 3 provides schematic illustrations and photographs of a printed product structure according to some embodiments.

FIG. 4A provides schematic illustrations and a photograph of a printed product comprising more than one drug according to some examples.

FIG. 4B is a schematic illustration of various printed product structures according to some examples.

FIG. 5 is a plot of differential scanning calorimetry results of pharmaceutical ingredients, extruded filaments, and printed products according to some examples.

FIG. 6 is a plot of powder X-ray diffraction results of pharmaceutical ingredients, extruded filaments, and printed products according to some examples.

FIG. 7 shows various polarized light microscopy images of pharmaceutical ingredients, extruded filaments, and printed products according to some examples.

FIG. 8 is a schematic illustration of manufacturing filaments according to some examples.

FIG. 9 is a plot of thermogravimetric analysis results of fenofibrate (FNB), hydroxpropylmethyl cellulose AS LG grade (HPMC AS LG), and a physical mixture (PM) of FNB and HPMC AS LG according to some examples.

FIG. 10 is a plot of differential scanning calorimetry (DSC) results of FNB, HPMC AS LG, a PM of FNB and HPMC AS LG, and an extruded filament (EXT) of FNB and HPMC AS LG according to some examples.

FIG. 11 is a plot of powder X-ray diffraction results of FNB, HPMC AS LG, a PM of FNB and HPMC AS LG, and an extruded filament (EXT) of FNB and HPMC AS LG according to some examples.

FIG. 12 is a plot of Fourier transform-infrared spectroscopic (FTIR) spectra of FNB, HPMC AS LG, a PM of FNB and HPMC AS LG, and an extruded filament (EXT) of FNB and HPMC AS LG according to some examples.

FIG. 13 shows various polarized light microscopy images of FNB, HPMC AS LG, a PM of FNB and HPMC AS LG, and an extruded filament (EXT) of FNB and HPMC AS LG according to some examples.

FIG. 14 shows various digital microscopy images of printed products according to some examples.

FIG. 15 shows breaking force test comparison setups of printed products according to some examples.

FIG. 16 shows images of printed products after breaking force testing according to some examples.

FIG. 17 shows breaking force test results of printed products according to some examples.

FIG. 18 shows in vitro drug release results of printed products according to some examples.

### DETAILED DESCRIPTION

The method and technology disclosed herein may be used in the manufacture of medications that include active ingredient(s), such as active pharmaceuticals, and excipients, such as polymers, plasticizers, inorganic carriers, etc. The disclosed techniques include those for preparing custom dosage forms of active pharmaceutical products, and the resultant products containing active pharmaceutical ingredients. Many compounding pharmacies prepare custom products containing active pharmaceutical ingredients, such as in the forms of flavored liquids, topical creams, transdermal gels, suppositories or other custom dosage forms. The techniques described herein provide for compounding of active pharmaceutical ingredients in precise and repeatable dosage forms using additive manufacturing technology.

Initially, filament forms of active pharmaceutical ingredients in a polymeric carrier, such as a biocompatible or digestible polymer, are created, which allow for precise control of the amount and distribution of the active pharmaceutical ingredients. Once the filaments are created, they can be used in an additive manufacturing process, such as a fused deposition method, where dosage forms are created in a layer-by-layer fashion as printed products.

Advantageously, the layer-by-layer fabrication processes described herein can use a continuous axis allowing for continuous or semi-continuous printing of multiple products in sequence to increase manufacturing throughput. For example, the products can be printed on a conveyor belt-type print bed, where the belt rotation direction corresponds, at least in part, to a vertical axis of the printed product. This can be achieved, in some examples, by positioning the deposition nozzle at an oblique angle to the print bed, such that the products can move away from the deposition nozzle by rotation of the belt as they are printed. Following printing, the products can be translated by the belt and be automatically removed from the belt as it rotates, where the products can be collected.

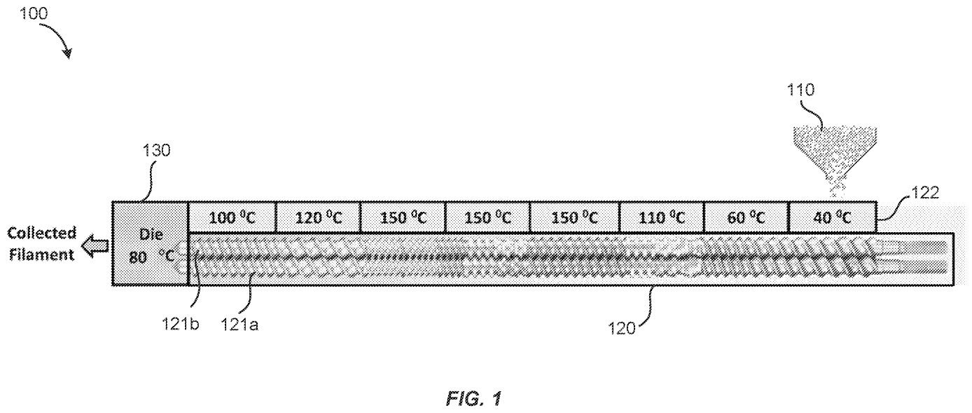

FIG. 1 is a schematic illustration of manufacturing filaments according to some examples. In some examples, device 100 is a hot-melt extruder that comprises a feeder (110), a barrel (120), screw(s) (121a, 121b), and a die (130). Referring to FIG. 1, materials can be manually, automatically, or mechanically fed into the barrel (120) through the feeder (110). Materials may include, but are not limited to, polymeric carriers, plasticizers, resins, active pharmaceutical ingredients (API), biologically active component of pharmaceutical products, or any mixtures. Exemplary materials that may be used as excipients include, but are not limited to, poly-vinylpyrrolidones (PVP), cellulose ethers, polyacrylates and polyacrylic acids. It will be appreciated that the use of different grades of polymers belonging to the above categories with different molecular weights and substitutions may be useful with different examples. In some examples, the feeder (110) may be a gravimetric or volumetric feeder. Exemplary APIs may include, but are not limited to, cardiovascular drugs such as aspirin, nifedipine, simvastatin, atorvastatin, anti-neoplastic drugs like temozolomide, or anti-diabetic drugs like dapagliflozin, metformin, semaglutide, etc.

The barrel (120) may be a chamber of any shape (e.g., cylindrical or the like). Furthermore, the barrel (120) may house or contain at least one feeder structure, thread, or screw (e.g., 121a, 121b) positioned in the barrel (120) extending substantially therethrough and a heater (122) that can increase and control the temperature of the barrel (120) to melt or soften the material fed into the barrel (120). Referring to FIG. 1, in some examples, more than one heater (122) may be arranged along the barrel (120) and the temperature of each heater may be individually controlled or adjusted. In some examples, the heater is a band heater that clamps around the chamber to provide uniform heating of the chamber. For example, when aspirin with a melting point of 135-138° C. is processed, the barrel temperature may be set at 150° C.

Referring to FIG. 1, materials enter the barrel (120) and come into contact with screws 121a and 121b. The screws (121a and 121b) force the fed materials forward into the heated barrel (120). Although FIG. 1 illustrates a twin-screw configuration, the number of possible screws can be housed in the barrel (120) is not limited to any specific number, and the barrel (120) can include any number of screws as desired. The screws (121a, 121b) may further comprise sub-screws of any dimensions, geometries, or configurations. In some embodiments, a Leistritz 12 mm twin screw that corotates within the barrel (120) is used. The screws (121a, 121b) may be driven and rotated by any appropriate means, such as an electric motor, hydraulic motor, or the like. In some examples, the rotating speed of each screw or sub-screw can be independently controlled and adjusted during the process. In a non-limiting example, a rotating speed of 120 rpm is used. As shown in FIG. 1, different dimensions and/or geometries of screws (121a, 121b) can be used, with different regions serving to crush, mix, feed, etc. the materials entering the barrel (120). Depending on the particular configuration, the screws (121a, 121b) can be used to create a homogeneous mixture or inhomogeneous mixture of the materials entering the barrel (120).

Materials forced through the barrel (120) by the screws (121a and 121b) then exit the barrel via the die (130). The die (130) forms the materials into the desired shape or geometry when the materials leave the barrel (120), which hardens and/or solidifies during cooling, forming a filament. In some examples, the filament can be cut to any desired length, can be wound on a spool, etc. In some examples, the filament can be stored in a sealed container to limit interaction with air, moisture, or the like.

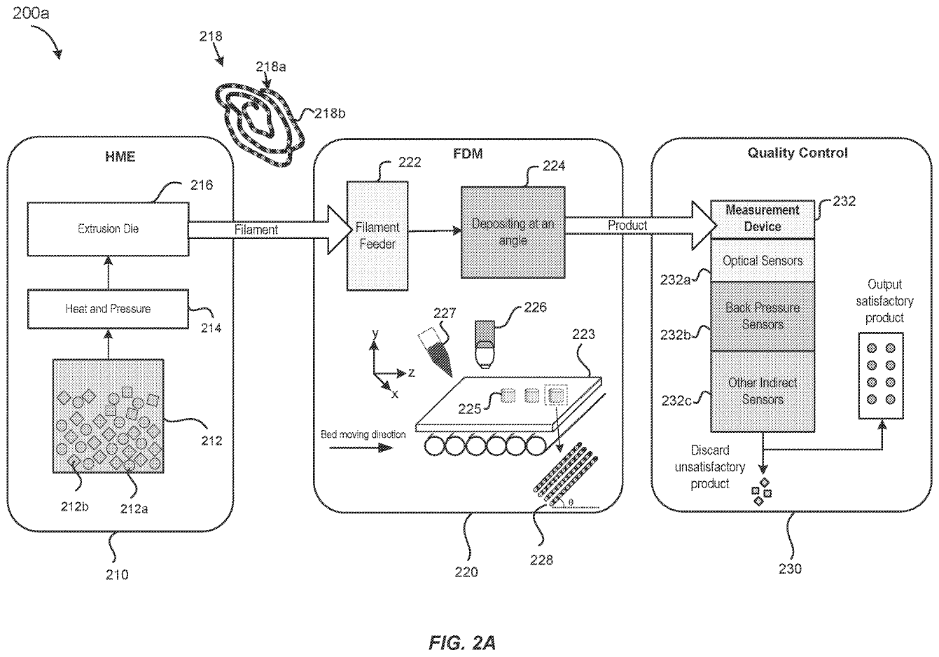

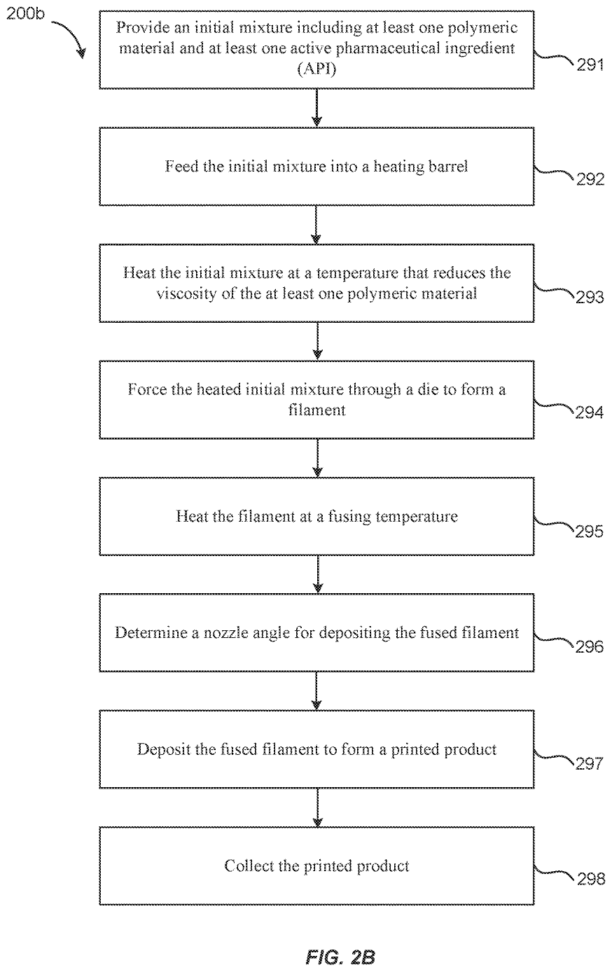

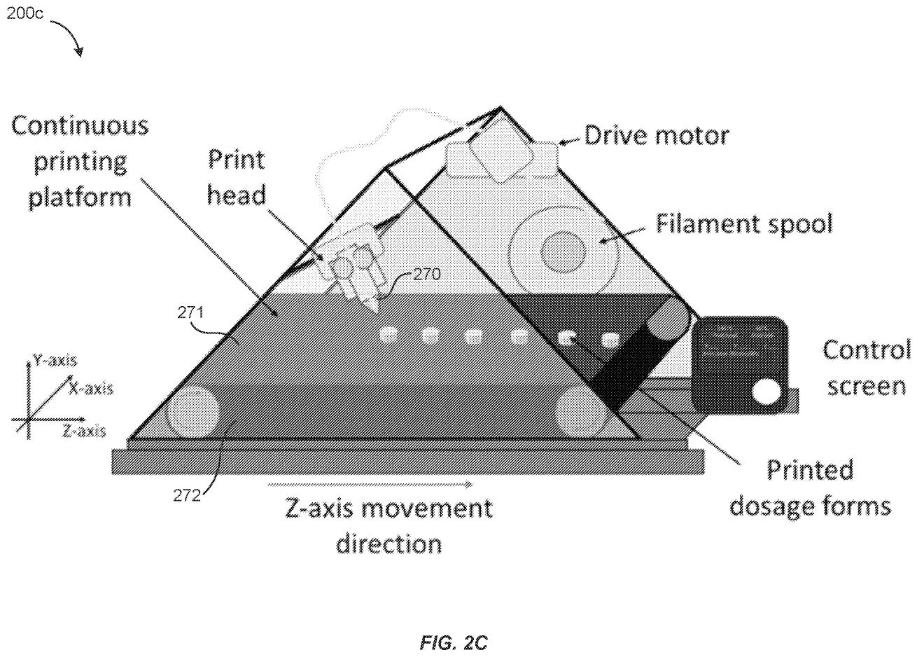

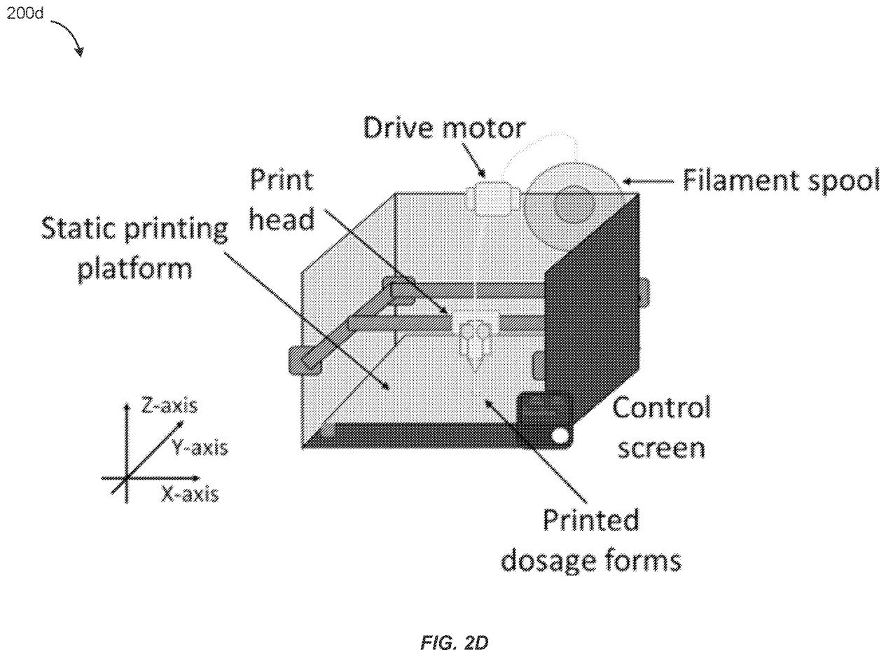

FIG. 2A is a block diagram 200a of manufacturing printed products according to some examples. API 212a and polymeric matrix material 212b are physically mixed to form a mixture 212 that can be fed into the hot melt extruder (HME) 210. Within the HME barrel, the mixture 212 is heated and pressurized at 214, such as by screws and/or heaters therein, and forced through the extrusion die 216 to form an extruded filament 218. In some embodiments, within the HME, the processing conditions (e.g., temperature, screw configuration, feed rate, screw speed, etc.) are maintained until the polymeric matrix material 212b and optionally the API 212a are molten and/or the API 212a solubilizes or is suspended or mixed in the polymeric matrix 212b. After cooling or any other optional processing steps, the processed mixture 212 exiting the die 216 forms extruded filament 218 comprising the API 218a in the polymetric matrix 218b, wherein the extruded filament 218 has properties (e.g., consistent diameter, flexibility, strength, etc.) suitable to be used as 3D printing filaments. The extruded filament 218 may contain the API in crystalline state, semi-crystalline state, or amorphous state. The extruded filament 218 may be fashioned into sections of any desired lengths and may optionally be wound around a spool.

In some embodiments, the extruded filament 218 can be fed to a fused deposition modeling (FDM) based 3D printing device 220 via the filament feeder 222. The FDM based 3D printing device 220 may be continuously connected with the HME 210 to form an integrated processing line for large-scale manufacturing, but this is not required in all examples, and filament 218 can be manually provided (e.g., as a spool or lengths of filament 218) to or as part of filament feeder 222. In some examples, the FDM based 3D printing device involves the additive deposition of molten feedstock or filament extruded through a computer-controlled deposition nozzle 226. The FDM based 3D printing device 220 can be capable of creating complex geometries as well as 3D models with controlled composition and architecture. In some examples, the FDM based 3D printing device 220 may comprise a hot-end part 224 that includes the computer-controlled deposition nozzle 226 and a relatively-cooler-end part that includes a build platform 223. To build a printed product 225, the FDM based 3D printing device 220 injects the molten filaments in a layer-by-layer fashion according to the structure and geometry of the printed product 225 while controlling position of the deposition nozzle 226 and build platform 223. The printed product 225 may have any shape or geometry as desired; possible shapes include, but are not limited to, cylindrical, cuboidal, caplet-like, torus-based, or film-based shapes. In some examples, the printed product 225 may be or comprise an amorphous solid dispersion (ASD). The ASD may include particles that further include API molecules dissolved in polymeric carriers and the particles form an amorphous, non-crystalline, structure. The formulation of an example ASD printed product may be the formulation as specified in Table 5 below. In some examples, the API, polymeric matrix, polymeric carrier, and/or plasticizer may be mixed using geometric dilution. In some examples, the API may be a Biopharmaceutical classification system (BCS) class II drug having high permeability and low solubility. In some examples, the polymeric matrix or polymeric carrier may comprise HPMC AS LG.

In some examples, when the extruded filament 218 enters the hot end part 224, the extruded filament 218 is heated to its transition temperature. As the extruded filament 218 becomes softened or molten, the viscosity of the filament is reduced. The molten filament 218 is then extruded through the computer-controlled deposition nozzle 226 onto the build platform 223. The computer-controlled deposition nozzle 226 may deposit the molten filament at different nozzle angles, which can provide for an unlimited dimension for continuous printing, such as where the build platform 223 is a conveyor belt, for example, as discussed below. The nozzle angle can be changed according to processing needs. In some embodiments, the nozzle angle is selected to be 45° to avoid excessive building of support layers. Printed product 228 shows an exemplary printed product built with a nozzle angle (θ) of 45°. In some examples, the nozzle angle may be set to an angle that is less than 45°. Furthermore, to diversify the materials that can be used, an extrusion syringe 227 along with the nozzle head may optionally be incorporated to the FDM based 3D printing device 220. The extrusion syringe 227 can be a semi-solid extrusion syringe that is capable of printing using gel or liquid-based materials that may be susceptible to thermal degradation. The extrusion syringe 227 may be actuated via a mechanical pump or any pressure-assisted mechanism, for example. Besides the extrusion syringe discussed above, any alternative kind of liquid dispenser may be used. The printed product 228 may be amorphous or crystalline. The API and the polymeric material of the printed product may be amorphous, semi-crystalline, or crystalline.

In some examples, the build platform 223 may be a dynamic platform such as a conveyor belt that moves toward the z-axis direction as the printing continues, or any similar configurations. The x-y plane defines the surface of the build platform 223. Although FIG. 2A illustrates a dynamic build platform 223 that moves unidirectionally along the z-axis, the dynamic build platform 223 may also move or shift the build platform toward more than one direction (e.g., toward the x-axis, the y-axis, or a combination of alternate movements toward the x-axis and the y-axis, etc.) when desired. Furthermore, as the computer-controlled deposition nozzle 226 may move along the x-axis, y-axis, or z-axis direction, the printed product 225 may be deposited at any location on the surface of the build platform 223, and the layout of the printed product 225 on the platform 223 is not limited to any specific arrangement of rows or columns. In some examples, as the printed product 225 can detach and fall off as they approach the end of the dynamic platform (e.g., the conveyor belt) due to the curling at the round end of the belt at the scraper, the printed product 225 can accordingly be removed and collected in an automated manner. This automated detachment reduces the chances of deformation after the application of force when the printed product 225 is removed manually. This automated detachment also makes this continuous fabrication process free from any manual intervention, which may further help comply the fabrication process with the regulatory guidelines and improve the healthcare space.

In some examples, a quality control block 230 may be integrated as an in-line monitoring block for optional downstream processing. Various characteristics, factors, or values of the printed product 225 may be monitored to ensure the product quality, reproducibility, and identify possible API degradation. An exemplary quality control block 230 may include optical sensors 232a that measure and interpret the electromagnetic spectra that result from the interaction between electromagnetic radiation and the printed product 225 as a function of the wavelength or frequency of the radiation. Exemplary optical sensors 232a include infrared (IR) spectroscopy, ultraviolet-visible-near-IR Spectroscopy (UV-Vis-NIR), Fourier transform infrared spectroscopy (FTIR), and the like. The optical sensors 232a may also include optical spectrometers (e.g., spectrophotometer, spectrograph, or spectroscope) that measure properties of light over a specific portion of the electromagnetic spectrum to identify materials and/or properties. In some embodiments, the optical sensors may be NIR fiber optic probes or the like.

In some examples, the quality control block 230 may further include back pressure sensors 232b to measure and monitor the force or pressure of molten filaments or fluids within the computer-controlled deposition nozzle 226 or the extrusion syringe 227 to ensure that the deposition is progressing properly.

In some examples, the quality control block 230 may also include other indirect sensors to measure various properties of the printed product of the printed product 225 and to monitor each stage of the manufacturing process. Properties may be measured and monitored include mass, density, material structure, and any other properties related to pharmaceutical tolerance or regulatory pharmaceutical values. The quality control block 230 can separate satisfactory printed product from unsatisfactory printed product according to various quality control factors. Satisfactory printed product can be output to the following processing stages like packing (not shown) while unsatisfactory product may be discarded or optionally recycled to HME 210.

FIG. 2B is a flowchart providing an overview of a method 200b of manufacturing printed products according to some examples. The method 200b includes, at 291, providing an initial mixture including at least one polymeric material and at least one active pharmaceutical ingredient (API). The polymeric material may be any substance or material that comprises macromolecules, wherein macromolecules can be composed of many repeating subunits. In some examples, the polymeric material can be, but is not limited to, thermoplastic polymers that have polymer chains connected by intermolecular forces, which weaken rapidly with increased temperature and yield a viscous liquid. Exemplary polymeric material includes hydroxypropyl cellulose (HPC), polyvinylpyrrolidone (PVP), polyvinyl alcohol (PVA), polylactic acid (PLA), etc. In some examples, the polymeric material may comprise polymeric carriers and plasticizers. Exemplary plasticizer includes Soluplus®, polyethylene glycols, Triacetin, etc. The API may be any component that provides biologically active or other direct effects in the diagnosis, cure, mitigation, treatment, or prevention of disease or to affect the structure or any function of the body of humans or animals. Exemplary APIs include, but are not limited to, acetylsalicylic acid (Aspirin), Nifedipine, chloroquine diphosphate, Ibuprofen, etc.

The method 200b further includes, at 292, feeding the initial mixture into a heating barrel of a hot-melt extruder (HME). As discussed above for FIG. 1, the barrel (120) may be a chamber of any shape (e.g., cylindrical and the like). Furthermore, the barrel (120) may house or contain at least one screw (e.g., 121a, 121b) positioned in the barrel (120) extending substantially therethrough and a heater (122) that can increase and control the temperature of the barrel (120) to melt or soften the material fed into the barrel (120). Referring to FIG. 1, in some embodiments, more than one heater (122) may be arranged along the barrel (120) and the temperature of each heater may be individually controlled or adjusted. In some embodiments, the heater is a band heater that clamps around the chamber to provide uniform heating of the chamber.

The method 200b further includes, at 293, heating the initial mixture at a temperature that reduces the viscosity of the at least one polymeric material. The method 200b further includes, at 294, forcing the mixture with reduced viscosity of the at least one polymeric material through a die to form a filament with desired shape and geometry. At 295, when the filament is fed into a deposition modeling (FDM) based 3D printing device, the filament is heated to a fusing temperature that softens or melts the fed filament. At 296, the nozzle angle for depositing the fused filament is determined or controlled according to the shape and geometry of desired printed products. The nozzle angle can be changed according to processing needs. In some embodiments, the nozzle angle is selected to be 45° to avoid excessive building of support layers. Printed product 228 shown in FIG. 2B shows an exemplary printed product built with a nozzle angle (θ) of 45°.

At 297, the fused filament is deposited, usually in a layer-by-layer manner, upon a build platform. Referring to FIG. 2A, the build platform 223 may be a dynamic platform such as a conveyor belt that moves or advances the z-axis direction as the printing continues, or any similar configurations. The x-y plane, in some cases, defines the surface of the build platform 223, but with the deposition nozzle 226 held at an angle, the surface of the build platform 223 can include components of z and x and/or y. For example, although FIG. 2A illustrates a dynamic build platform 223 that moves unidirectionally along the z-axis, the dynamic build platform 223 may also move or shift the build platform toward more than one direction (e.g., toward the x-axis, the y-axis, or a combination of alternate movements toward the x-axis and the y-axis, etc.) when desired. Furthermore, as the computer-controlled deposition nozzle 226 may move along the x-axis, y-axis, or z-axis direction, the printed product 225 may be deposited at any location on the surface of the build platform 223, and the layout of the printed product 225 on the platform 223 is not limited to any specific arrangement of rows or columns. Advantageously, positioning the deposition nozzle 226 at an angle to the build platform 223 allows the z-axis to vary continuously and indefinitely, allowing for products of unlimited length to be fabricated or for continuous or semi-continuous fabrication of products without interruption. Comparisons between an example FDM-based 3D printing system including the dynamic platform the and a conventional (e.g., batch process based) FDM-based 3D printing system are illustrated in FIGS. 2C and 2D.

The method 200b further includes, at 298, collecting the printed product. The method 200b is not limited to the steps described in the flowchart. In some examples, additional nozzles may be used and each additional nozzle may deposit filaments comprising different APIs or polymeric material. For example, referring to FIG. 2A, an extrusion syringe that is capable of printing using gel and lipid-based materials may be used. Furthermore, the method 200b may include any quality control steps as desired. Exemplary quality control characteristics or values include, but are not limited to, the mass, density, and material structure of the printed product.

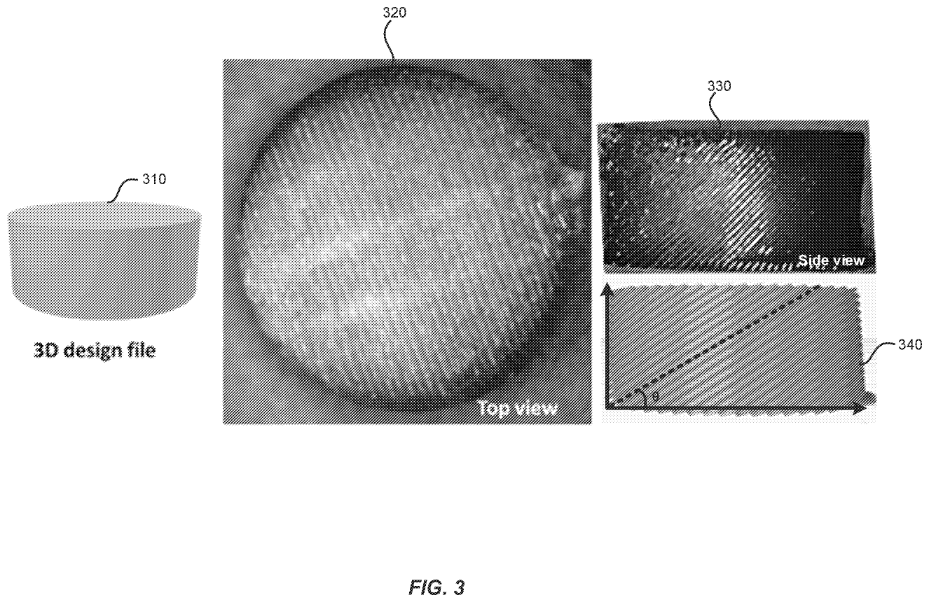

FIG. 2C is an example FDM-based 3D-printing system including a dynamic platform according to some examples. As discussed in relation to FIGS. 2A and 3, the example printing system 200c may include a dynamic platform that moves continuously in the z-axis as printing progresses. In some examples, the extrusion nozzle 270 of the printhead may be oriented at an angle less than 90° to a build surface 271, which bypasses the pre-print and post-print lag times and reduces manual intervention. In some examples, the dynamic platform may be a conveyor belt 272 that moves along the z-axis at a specified angle to minimize or eliminate the use of support structures during printing. In some examples, the angle between the printhead and the build surface 271 may be 20-30°, 30-40°, 40-50°, 50-60°, or 60-70°.

FIG. 2D is an example conventional FDM-based 3D-printing system according to some examples. Compared with the example system 200c in FIG. 2C, the example conventional FDM-based 3D-printing system 200d does not include a dynamic platform that moves continuously in the z-axis as printing progresses. In some examples, the system and its corresponding printing method described in FIG. 2D may be referred to as “batch printing system” and “batch printing method”.

FIG. 3 is a schematic illustration of a printed product structure according to some embodiments. The 3D design file 310 of the printed product may be created by computer-aided design (CAD) using any suitable CAD software. A photograph of the top view of the printed product is depicted as 320 and a photograph of the side view of the printed product is depicted as 330. As illustrated by the sliced 3D design file 340 of the printed product, the orientation of each deposited layer of the printed product may be at an angle θ in some embodiments (e.g.,) 45°. The orientation of each deposited layer may be of any angle according to the sliced 3D design file 340. Furthermore, the thickness of each deposited layer as well as the infill amount may be different. In some embodiments, the infill amount of each layer varies based on the desired porosity of the printed product. For example, a completely solid product may have an infill amount of 100%. On the other hand, as higher porosity may be desired to ensure satisfactory drug release, a printed product with higher porosity may have a lower infill amount percentage than a completely solid product.

Tables 1-4 provide various exemplary formulations of printed products.

TABLE 1
Exemplary Formulation of Printed Product I.
Formulation

| Ingredient | Purpose | Weight % |
|---|---|---|
| Acetylsalicylic acid (Aspirin) | API | 20 |
| Hydroxypropyl cellulose | Polymeric matrix | 40 |
| Soluplus | Plasticizer | 40 |

TABLE 2
Exemplary Formulation of Printed Product II.
Formulation

| Ingredient | Purpose | Weight % |
|---|---|---|
| Nifedipine | API | 10 |
| Hydroxpropylmethyl | Polymeric matrix | 80 |
| cellulose AS MG | | |
| Polyethylene oxide | Plasticizer | 10 |

TABLE 3
Exemplary Formulation of Printed Product III.
Formulation

| Ingredient | Purpose | Weight % |
|---|---|---|
| Chloroquine diphosphate | API | 20 |
| Hydroxypropyl cellulose | Polymeric matrix | 40 |
| Soluplus | Plasticizer | 40 |

TABLE 4
Exemplary Formulation of Printed Product IV.
Formulation

| Ingredient | Purpose | Weight % |
|---|---|---|
| Ibuprofen | API | 20 |
| Hydroxpropylmethyl | Polymeric matrix | 80 |
| cellulose AS | | |

TABLE 5
Exemplary Formulation of Printed Product V.
Formulation

| Ingredient | Purpose | Weight % |
|---|---|---|
| fenofibrate (FNB) | API | 20 |
| Hydroxpropylmethyl | Polymeric carrier | 80 |
| cellulose AS | | |

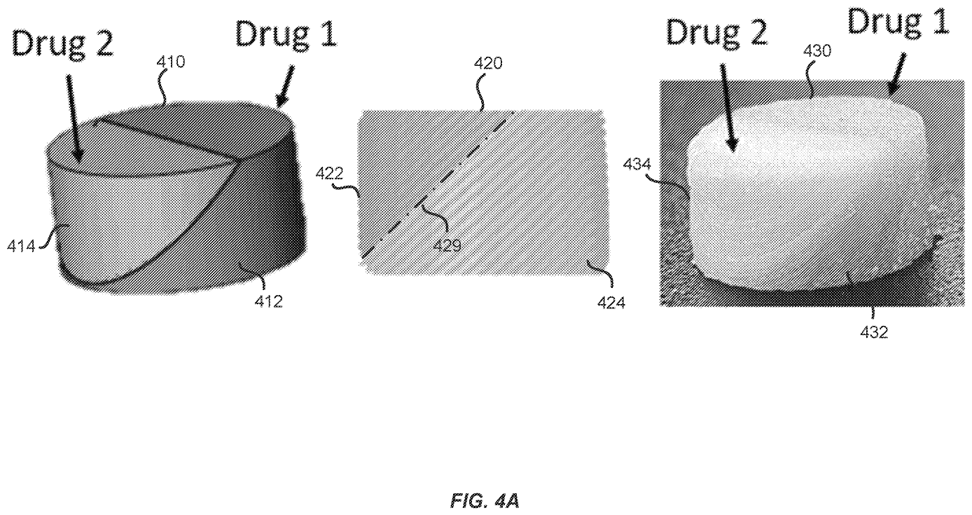

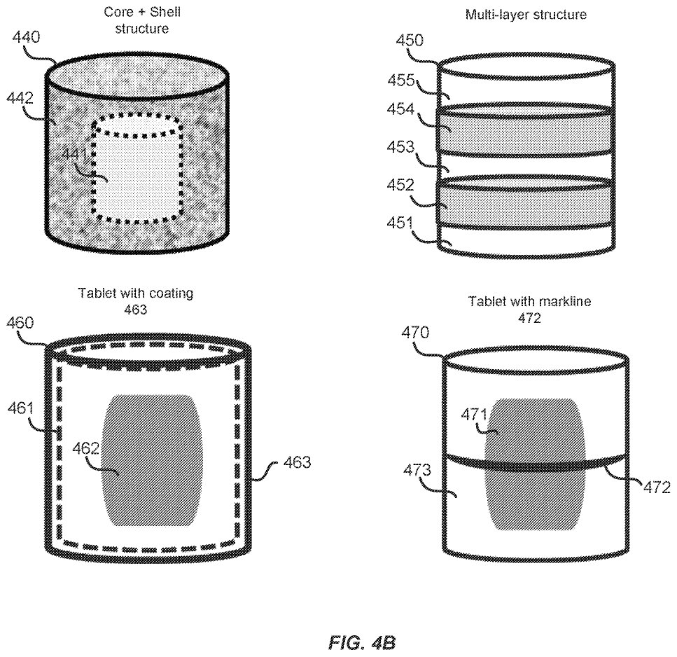

FIG. 4A is a schematic illustration of a printed product comprising more than one drug according to some examples. The 3D design file 410 of the printed product illustrates a first drug segment 412 and a second drug segment 414. Each of the drug segments 412 and 414 may further comprise at least one polymeric material and at least one API. As illustrated by the sliced 3D design file 420 of the printed product, the first drug segment and the second drug segment may be separated at an interface line 429 where two adjacent layers contain different type of drug segments (e.g., the first drug segment 422 and the second drug segment 424). The percentage of each drug segment may be adjusted when different drug characteristics are desired. For example, when a higher dosage of the API included in the first drug segment is preferred, printed product may be designed to comprise the first drug segment in a higher percentage (e.g., weight percentage, volume percentage, etc.). A photograph of an exemplary printed product 430 comprising the first drug segment 432 and the second drug segment 434 is shown for illustration purposes.

FIG. 4B is a schematic illustration of various printed product structures according to some examples. Printed product 440 comprises a core part 441 and a shell part 442 inside core part 441. In some embodiments, the core part 441 can be a first drug segment that further comprises a first API and a polymeric material. Additionally, the core part 441 is not limited to 3D printed drug segments and the core part 441 can be or may include a liquid- or gel-based API that is injected via a syringe or any similar device. The shell part 442 may be a compartment that houses the first drug segment to control the release of the API in the first drug segment. The shell part 442 may also be a second drug segment that further comprises a second API and/or a second polymeric material. In some cases, the shell part 442 may provide controlled release of core part 441 after some time period or under certain conditions; for example, shell part may comprise material that can break down only under certain pH conditions, such as to ensure release of core part 441 in a particular part of the digestive system. Printed product 440 is for illustration purposes only. The configuration of the core part and the shell part is not limited to any specific design. It will be appreciated that a printed product may comprise more than one core part and/or shell part.

Printed product 450 has a multi-layer structure including layer 451, layer 452, layer 453, layer 454, and layer 455. Each of layer 451, layer 452, layer 453, layer 454, and layer 455 may individually be a drug segment that further comprises at least one API and/or a polymeric material, a polymeric material layer, an API layer, a coating layer such as a sugar coating layer to disguise the taste of the API, a release control coating layer to delay the release of the API, or any substance or materials as desired. Additionally, each layer (e.g., layer 451, layer 452, layer 453, layer 454, and layer 455) is not required to be 3D printed, and each layer can be or may further include liquid or gel that is injected via a syringe or any similar device. It will be appreciated that the number, the shape or geometry, the arrangement, and the sequence of the layers are not limited to the structure described as printed product 450.

Printed product 460 comprises a core part 462, a shell part 461, and a coating part 463 encapsulating the shell part 461. Each of the core part 462, the shell part 461, and the coating part 463 may optionally be a drug segment that comprises at least one API and/or a polymeric material, a polymeric material layer, an API layer, or any substance or materials as desired. The coating part 463 may be any coating layer as desired. Exemplary coating part 463 includes a sugar-coating layer to disguise the taste of the API, a release control coating layer to delay the release of the API, or the like.

Printed product 470 comprises a core part 471, a shell part 473, and a markline 472 visibly embedded on the surface of the shell part 473. The markline 472 may be deposited as a very thin layer that forms a slice of the shell part 473. In some embodiments, the markline 472 may also be deposited directly on the surface of the shell part 473 by using 3D printing or any similar depositing technology. Printed product 470 may include more than one markline and the marklines may be arranged in any pattern for aesthetic, marking, or any purposes as desired. In some examples, the markline may correspond to a recessed region in printed product 470.

Although printed products 440, 450, 460, and 470 depict printed products in cylindrical tablets or elongated tablets, the shape or geometry of a printed product is not limited to the examples depicted, and irregular or complex shapes, such as donut shape, star shape, heart shape, or the like, can be used. For example, the techniques describe herein may also be used to make different printed products including APIs in any desirable form or shape, such as thin-films, microneedles, etc.

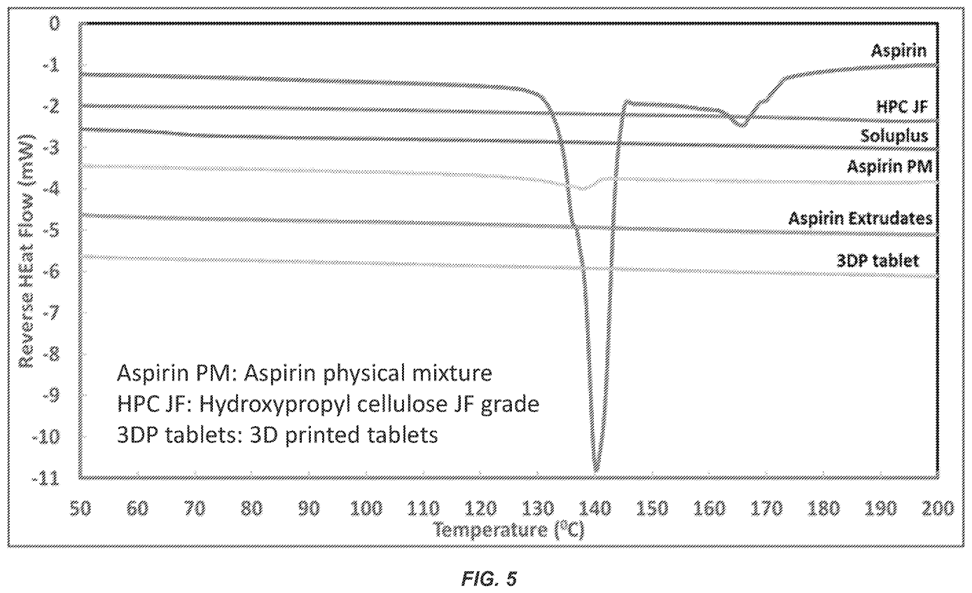

FIG. 5 is a plot of differential scanning calorimetry results of pharmaceutical ingredients, extruded filaments, and printed products according to some examples. The schematic illustration provides that the processing conditions are maintained such that the API in the printed product is completely rendered amorphous after the process. In some cases, analysis by differential scanning calorimetry can confirm components and/or amounts of components in a printed product.

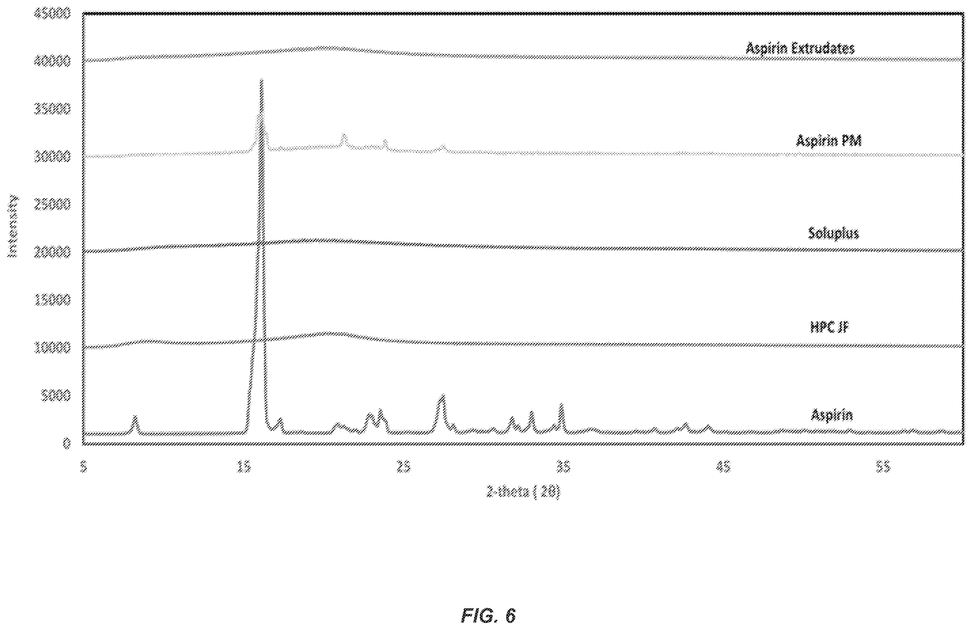

FIG. 6 is a plot of powder X-ray diffraction results of pharmaceutical ingredients, extruded filaments, and printed products according to some examples. The results show that the processing conditions are maintained such that API in the printed product is completely rendered amorphous after the process. In some cases, analysis by powder X-ray diffraction can confirm components and/or amounts of components in a printed product.

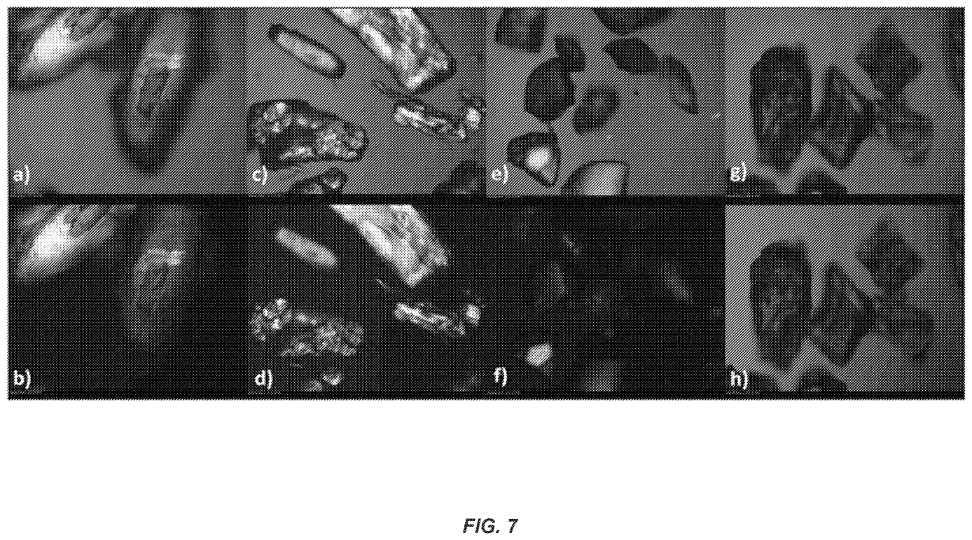

FIG. 7 shows various polarized light microscopy images of pharmaceutical ingredients, extruded filaments, and printed products according to some examples. FIG. 7 panel a) shows a color polarized light microscopy image of pure Aspirin and FIG. 7 panel b) shows a black-white polarized light microscopy image of pure Aspirin. FIG. 7 panel c) shows a color polarized light microscopy image of an exemplary Aspirin mixture and FIG. 7 panel d) shows a black-white polarized light microscopy image of an exemplary Aspirin mixture. FIG. 7 panel e) shows a color polarized light microscopy image of a filament including Aspirin as an API according to some embodiments and FIG. 7 panel f) shows a black-white polarized light microscopy image of a filament including Aspirin as an API according to some embodiments. FIG. 7 panel g) shows a color polarized light microscopy image of a printed product including Aspirin as an API according to some embodiments and FIG. 7 panel h) shows a black-white polarized light microscopy image of a printed product including Aspirin as an API according to some embodiments.

Aspects of the invention may be further understood by the following non-limiting examples.

### Example 1

An exemplary formulation of a printed product includes Nifedipine, hydroxpropylmethyl cellulose AS MG (HPMC AS, MG grade; HPMC AS MG), and polyethylene oxide (molecular weight 6000) (PEG 6K). By using hot-melt extrusion process, the API Nifedipine with low solubility and photostability is converted into an amorphous solid dispersion that is suitable for FDM based 3D printing process. The exemplary formulation of the printed product is summarized in Table 6.

TABLE 6
Formulation

| Ingredient | Purpose | Weight % |
|---|---|---|
| Nifedipine | API | 10 |
| Hydroxpropylmethyl | Polymeric matrix | 80 |
| cellulose AS MG | | |
| Polyethylene oxide | Plasticizer | 10 |

Here, the drug load of the printed product may be set at 30% to provide flexibility to dose personalized medicines in line with the commercially available dosing for patients. In combination with the improved solubility of the amorphous solid dispersion, the corresponding drug dosing and performance of the printed product can be further improved. The processing condition of the hot-melt extrusion process for the exemplary formulation is illustrated in FIG. 1. The processing conditions of the FDM based 3D printing process of a printed product with the exemplary formulation is summarized in Table 7.

TABLE 7

| Printing parameters | Values | |
|---|---|---|
| Print temperature | 150° | C. |
| Bed temperature | 60° | C. |
| Print speed | 40 | mm/s |
| Layer height | 0.1 | |
| Wall thickness | 0.4 | mm |
| Infill pattern | Grid | |
| Infill density | 80% | |
| Top/Bottom layer | 0.8 | mm |

| | |
|---|---|
| Print nozzle core | AA 0.4 |

### Example 2

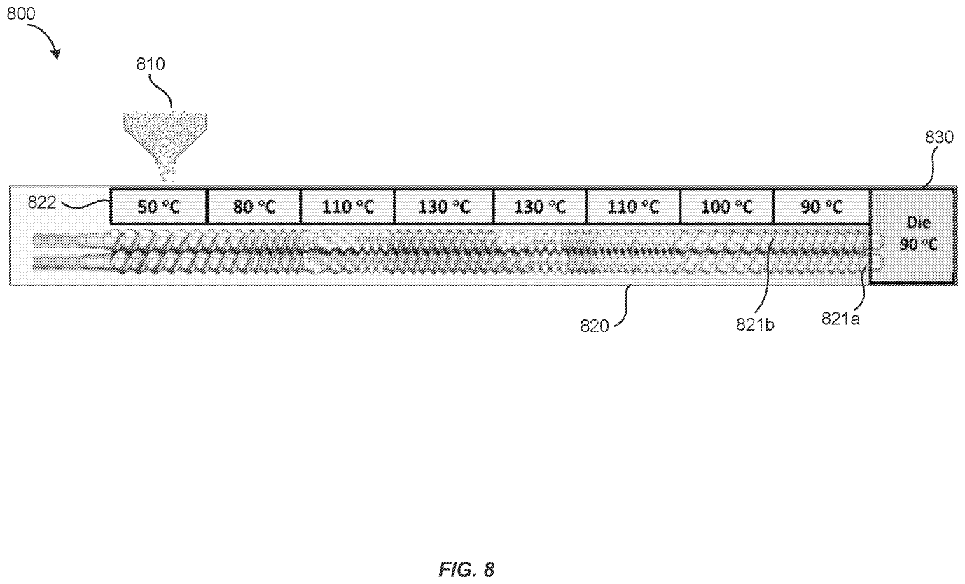

An exemplary formulation of an ASD printed product includes FNB, hydroxpropylmethyl cellulose (HPMC) AS LG. The FNB and HPMC AS LG may be mixed using geometric dilution. The exemplary formulation of the ASD printed product is summarized in Table 8. By using hot-melt extrusion (HME) process, filaments for 3D printing including FNB and HPMC AS LG are fabricated. Example HME temperature profile and screw design for the extrusion process are shown in FIG. 8.

TABLE 8
Formulation

| Ingredient | Purpose | Weight % |
|---|---|---|
| FNB | API | 20 |
| Hydroxpropylmethyl | Polymeric carrier | 80 |
| cellulose AS LG | | |

FIG. 8 is a schematic illustration of a system for manufacturing filaments according to some examples. The blend of FNB and HPMC AS LG may be introduced into a feeder 810 of a HME device 800. As illustrated by FIG. 8, the HME device 800 may include the feeder 810, a barrel 820, screw(s) (821a, 821b), and a die 830. The barrel 820 may be a chamber of any shape (e.g., cylindrical or the like). The barrel 820 may house or contain at least one feeder structure, thread, or screw (e.g., 821a, 821b) positioned in the barrel 820 extending substantially therethrough and a heater 822 that can increase and control the temperature of the barrel 820 to melt or soften the material fed into the barrel 820. Referring to FIG. 8, in some examples, more than one heater 822 may be arranged along the barrel 820 and the temperature of each heater may be individually controlled or adjusted. In some examples, the feeder 810 may be a calibrated volumetric feeder and the blend is introduced into the HME device 800 at a feeding rate of 3 g/min. In some examples, the extrusion process may be run at 50 rpm. In some examples, the die 830 may have a geometry and/or size that match the requirements of the FMD printer being used in any following processing steps. In some examples, the die 830 may have a size (e.g., diameter) of 1.75 mm. In some examples, the die pressure may be 60±10 psi at equilibration and the torque may be 4.80±0.57 N·m at equilibration; the die pressure and the torque may be maintained at a steady rate. In some examples, the size of the extruded filaments may be monitored by using any mechanism or apparatus as desired. In some examples, a Vernier caliper may be used to monitor the size of the extruded filaments. In some examples, the extruded filaments may be collected and stored in a validated desiccator for following use and characterization. The temperature ranges as illustrated in FIG. 8 may be used to ensure the extruded filaments (e.g., filaments including FNB and HPMC AS LG) have a satisfactory balance between flexibility and brittleness for following processing steps (e.g., FDM-3D printing). The extruded filaments have a uniform diameter distribution that does not vary along the filament length.

TABLE 8
Formulation

| Ingredient | Purpose | Weight % |
|---|---|---|
| FNB | API | 20 |
| Hydroxpropylmethyl | Polymeric carrier | 80 |
| cellulose AS LG | | |

The processing conditions of the FDM based 3D printing process of the ASD printed product with the exemplary formulation is summarized in Table 9. In some examples, the printed product may have a cylindrical shape with an 8-mm diameter and 5-mm height. Infill density for printing may be 10-20%, 20-30%, 30-40%, 40-50%, 50-60%, 60-70%, and 70-80%. In some examples, the geometry of the printed product may be sliceable or divisible into subcomponents by a software, an algorithm, or a machine learning model. In some examples, the printed product is divided into a plurality of layers and each layer has a thickness of 0.1 mm.

TABLE 9

| Printing parameters | Values | |
|---|---|---|
| Print temperature | 140° | C. |
| Bed temperature | 60° | C. |
| Print speed | 20 | mm/s |
| Layer thickness | 0.1 | mm |

| | |
|---|---|
| Infill pattern | Grid |
| Infill density | 25%, 50%, or 75% |
| Print nozzle core | AA 0.4 |

In some examples, a 1.75-mm round-shaped die may be used to fabricate FNB-loaded filaments. The collected filaments may have a diameter of 1.65±0.05 mm. The diminished diameter of collected filaments may be attributed to the thinning caused by the forces being applied by the puller during collection as well as the immediate swelling of the filaments post-heating from the extrusion process followed by the contraction on cooling. The swell ratio increases as the processing temperature and the temperature of the extrudates at the outlet increase.

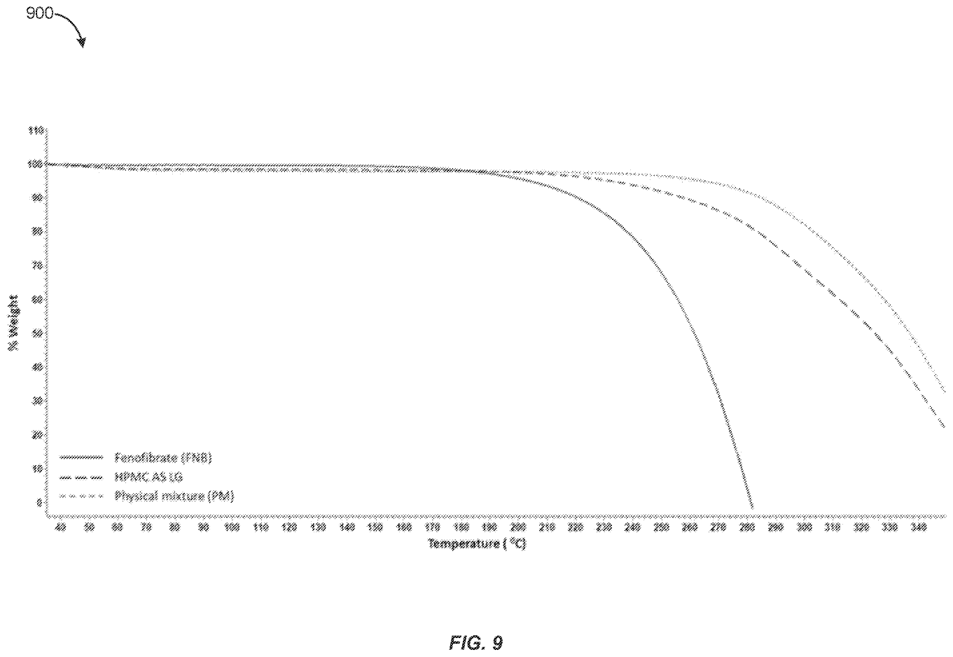

FIG. 9 is a plot of thermogravimetric analysis (TGA) results of fenofibrate (FNB), HPMC AS LG, and a physical mixture (PM) of FNB and HPMC AS LG according to some examples. Thermogravimetric analysis may be performed to understand the thermal properties of the pure crystalline drug or API (e.g., FNB), polymeric matrix or polymeric carrier (e.g., HPMC AS LG), and the physical mixture of the API and the polymeric matrix or polymeric carrier. In some examples, the analysis samples may be loaded into an open crucible, placed in the furnace, and heated from 35 to 350° C. at a rate of 10° C./min. The TGA may be run under an ultra-purified nitrogen environment at a 50 mL/min purge gas flow rate. The data may be collected and analyzed using software or an algorithm. The TGA curves of FNB, HPMC AS LG, and the PM of FNB and HPMC AS LG are shown in solid line, dashed line, and dotted line, respectively.

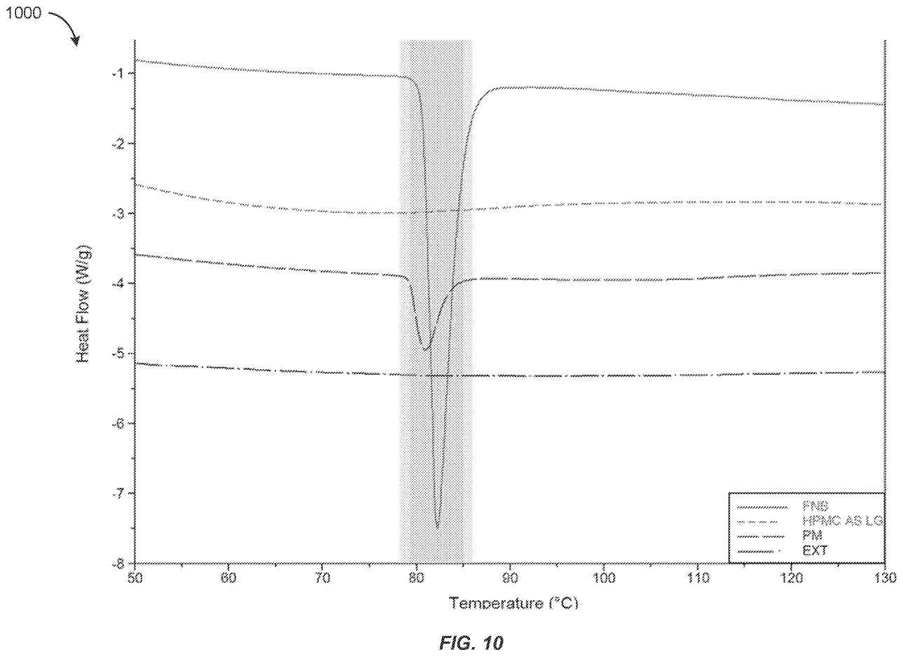

FIG. 10 is a plot of differential scanning calorimetry (DSC) results of FNB, HPMC AS LG, a PM of FNB and HPMC AS LG, and an extruded filament (EXT) of FNB and HPMC AS LG according to some examples. DSC analysis may be conducted to determine the amorphization of FNB under corresponding processing conditions. In some examples, HPMC AS LG may be used as polymeric matrix acting as the dispersion medium to solubilize FNB when HPMC AS LG is in the molten state in the extrusion process. As illustrated by FIG. 10, the melting of pure FNB samples and PM samples start at 79° C. and 78° C., respectively. The regions highlighted under blue and red bars indicate the melting regions for pure FNB samples and PM samples, respectively. The region highlighted under purple (e.g., the common region between the blue and red bars) displays a depression of melting point for pure FNB samples, which does not appear to be significant, given the closeness of values (e.g., 79° C. and 78° C.) as mentioned above. The glass transition temperature of the polymeric matrix (e.g., HPMC AS LG) is observed between 12° and 130° C. The DSC results may determine the processing parameters (e.g., screw design, thermal profile, etc.) for the HME process being used to make FNB-loaded filaments as discussed in relation to EXAMPLE 2 above. As illustrated by FIG. 10, no endothermic peaks corresponding to the melting peak of FNB appear in the plot of EXT, indicating that FNB has dissolved or dispersed in the polymeric matrix (HPMC AS LG) before reaching its melting points. The absence of FNB endothermic peaks may also indicate that the mixture of FNB and HPMC AS LG is converted to its amorphous state during the extrusion process.

The compatibility of drug-polymer miscibility may be evaluated by applying the theoretical structural orientation-based prediction model of Hansen Solubility Parameters (Δδ=3.40 MPa^{1/2} e.g., <7) of FNB (δt=20 MPa^{1/2}) and HPMC-AS polymer (δt=24 MPa^{1/2}) (where δ(MPa^{1/2}) is the (total) solubility parameter). Based on the Δδ values, it can be confirmed that FNB and HPMC AS LG are highly likely to be miscible and would form a solid dispersion. This theoretical evaluation of miscibility during the pre-formulation stages is important to predict the possibility of converting a crystalline drug to its amorphous state to form an amorphous solid dispersion (ASD). The balance needs to be achieved between the intramolecular interaction energy within a drug and the intermolecular drug-polymer interactions, where the polymer acts as the carrier matrix in this case.

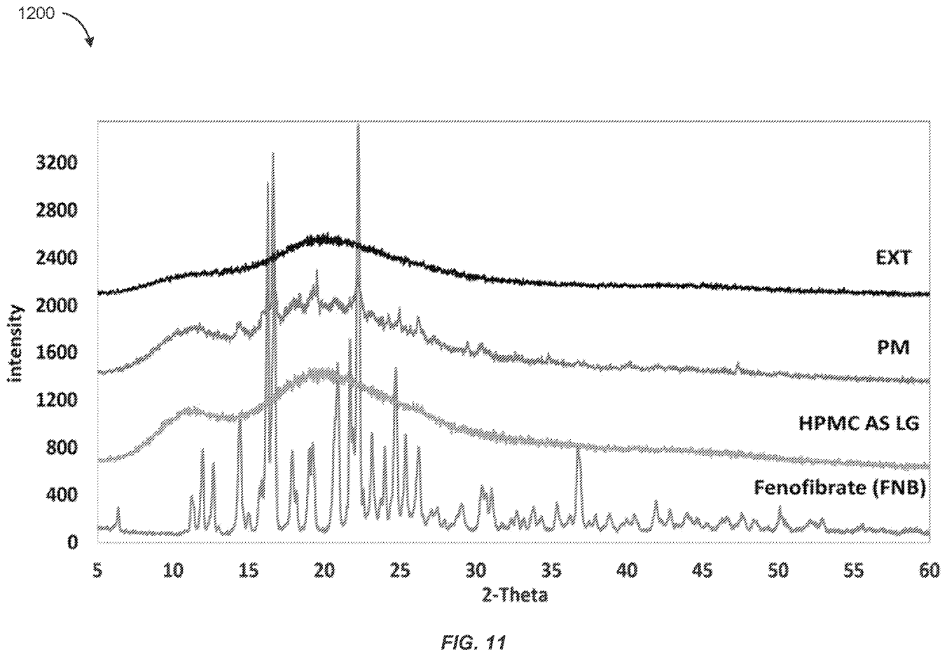

FIG. 11 is a plot of powder X-ray diffraction (XRD) results of FNB, HPMC AS LG, a PM of FNB and HPMC AS LG, and an extruded filament (EXT) of FNB and HPMC AS LG according to some examples. The samples (pure crystalline FNB, HPMC AS LG, PM, and EXT crushed into fine powder) are evenly spread into the XRD holder and analyzed over a 20 range of 5°-60° with a scan speed of 2°/min and a step size of 0.02°/min. The crystalline characteristics of pure FNB can be observed at distinct peaks at a 2-theta of 16.26°, 16.70°, 22.2°, 24.7°, and 47.7°. HPMC AS LG shows no distinct peaks and exhibits the distinct halo corresponding to its amorphous nature. The PM showed the same distinct peaks that were exhibited by pure FNB, but with reduced intensity indicating the crystalline nature of the drug in the PM before processing. The EXT after extrusion does not show the presence of peaks corresponding to pure FNB and exhibits a halo corresponding to amorphous nature, thereby indicating that the EXT's amorphization of FNB in the polymeric matrix (e.g., HPMC AS LG).

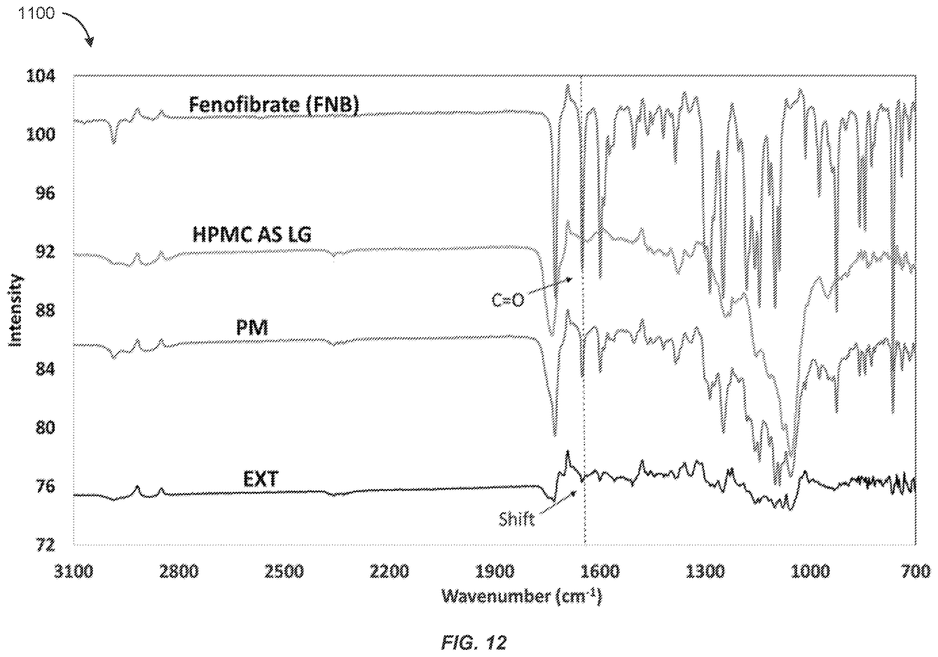

FIG. 12 is a plot of Fourier transform-infrared spectroscopic (FTIR) spectra of FNB, HPMC AS LG, a PM of FNB and HPMC AS LG, and an extruded filament (EXT) of FNB and HPMC AS LG according to some examples. FTIR analysis may be performed to determine the intramolecular interactions between FNB and HPMC AS LG in the extruded filaments after confirming the formation of ASD. As FIG. 12 illustrates, pure FNB spectrum shows peaks at around 1650 cm⁻¹ correspond to C═O stretching because of the ester group. These peaks can also be seen in the PM and EXT spectra with reduced intensity. The EXT spectrum shows a slight shift towards higher wavenumber for these peaks, which may be caused by the interaction between the API (e.g., FNB) and the polymeric matrix (e.g., HPMC AS LG). The interaction may be contributing to the stabilization of ASD.

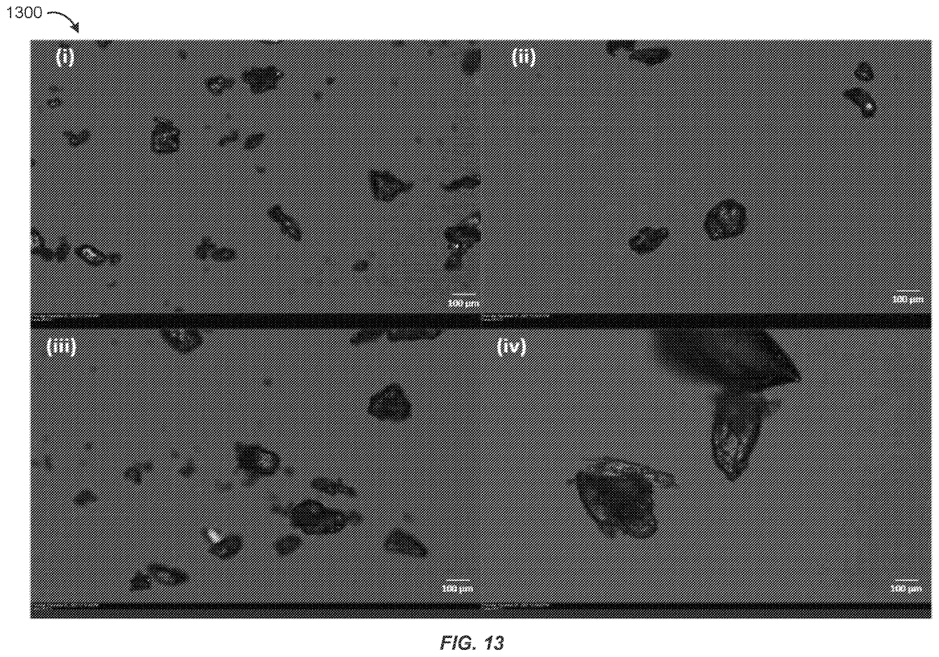

FIG. 13 shows various polarized light microscopy (PLM) images of FNB, HPMC AS LG, a PM of FNB and HPMC AS LG, and an extruded filament (EXT) of FNB and HPMC AS LG according to some examples. The birefringence in crystalline substances (e.g., pure FNB) may be observed under 10× magnification. PLM analysis may be used to observe the distribution of the API (e.g., FNB) in the polymeric matrix (e.g., HPMC AS LG) and any traces of crystallinity in the extruded filaments. As FIG. 13 shows, bulk FNB exhibits birefringence due to its crystalline nature, which gives it the property to refract light. HPMC AS LG shows light polarization, which might be resulted from its semi-crystalline structures. The birefringent pattern is observed in the PM sample but not the EXT sample, indicating that the absence of crystallinity in the processed filaments (e.g., EXT). The traces of birefringence observed in the ASD including FNB and HPMC AS LG are due to the semi-crystalline backbone of the polymeric matrix HPMC AS LG.

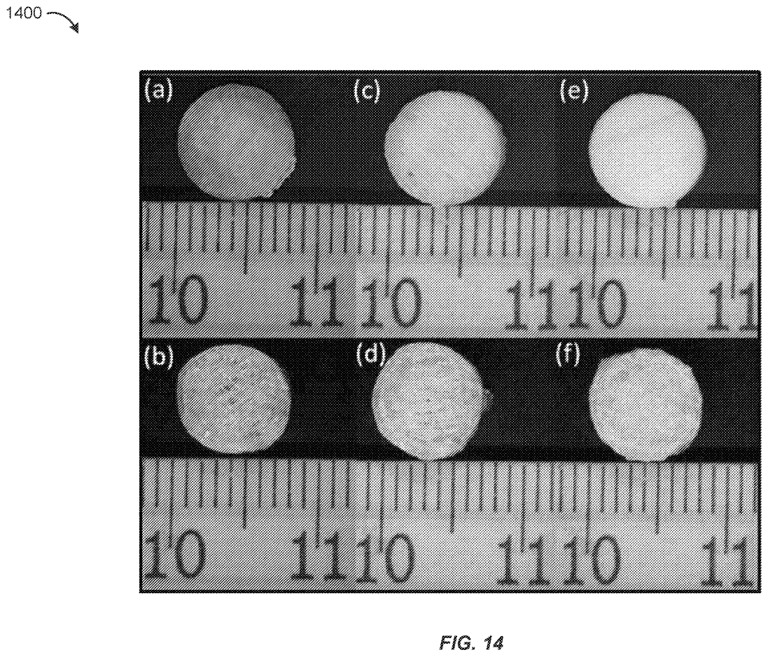

FIG. 14 shows various digital microscopy images of printed products according to some examples. Printed products fabricated using the continuous printing method (e.g., methods including the dynamic platform as discussed in relation to FIGS. 2A and 2C) have smoother and more regular surface as compared to printed products fabricated using the batch printing method (e.g., conventional FDM-based 3D-printing methods as discussed in relation to FIG. 2D). Image (a) shows a sample printed product of 25% infill fabricated by continuous printing method. Image (b) shows a sample printed product of 50% infill fabricated by continuous printing method. Image (c) shows a sample printed product of 75% infill fabricated by continuous printing method. Image (d) shows a sample printed product of 25% infill fabricated by batch printing method. Image (e) shows a sample printed product of 50% infill fabricated by batch printing method. Image (f) shows a sample printed product of 75% infill fabricated by batch printing method.

Printed products fabricated using the continuous printing method show higher structural integrity and adherence to a target shape. The structural integrity and adherence to a target shape for printed products may be characterized by dimensional measurements. The dimensional measurements of printed products (n=10) printed using continuous printing process (C) and batch printing process (M) is summarized in Table 10.

TABLE 10

| Process | Diameter (mm) | Height (mm) |
|---|---|---|
| Batch printing | 7.843 ± 0.294 | 4.967 ± 0.199 |
| Continuous | 8.007 ± 0.055 | 5.004 ± 0.018 |
| printing | | |

The visually-observed quality of the printed products fabricated by continuous printing method is higher than the quality of the printed products fabricated by batch printing method. In some examples, the initial bottom layers of the printed product fabricated by batch printing method at a 0° axis may have an increased circumference and a decreased layer thickness. The increased circumference and decreased layer thickness may be caused by the pressure exerted on these initial bottom layers as well as gravitational force created by the upper layers built on top of these bottom layers, which leads to irregularity in the printed product. Compared with printed products fabricated by batch printing method, printed products fabricated by continuous printing method do not show similar irregularity. As the continuous printing method allows printed products to move forward and make space for the next layer to be printed along a 45° axis, the continuous printing method avoids direct exertion of pressure on lower printed layers during printing.

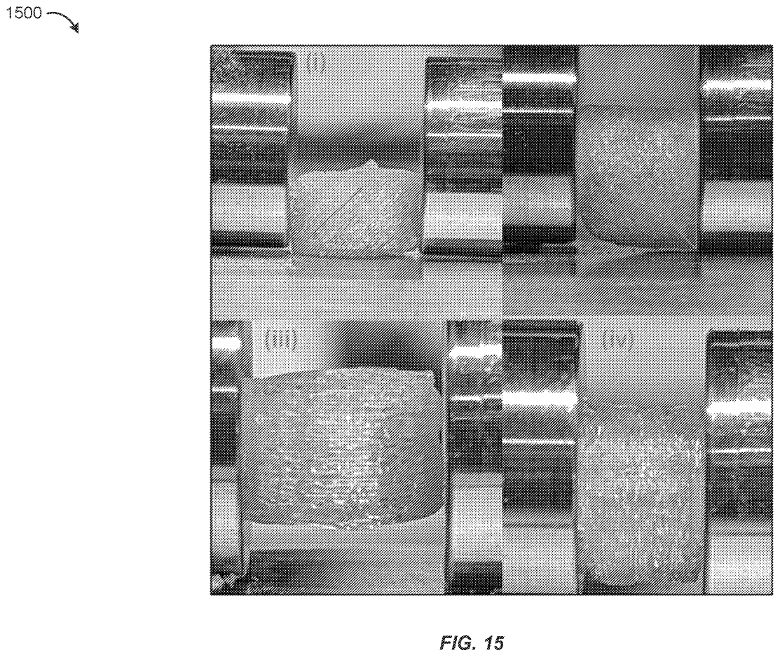

FIG. 15 shows breaking force test comparison setups of printed products according to some examples. The printed products may be placed horizontally for testing. Images (i)-(iv) show various testing setups. For examples, image (i) shows an example horizontal testing setup for a printed product fabricated by continuous printing method. Image (ii) shows an example vertical testing setup for a printed product fabricated by continuous printing method. Image (iii) shows an example horizontal testing setup for a printed product fabricated by batch printing method. Image (iv) shows an example vertical testing setup for a printed product fabricated by batch printing method.

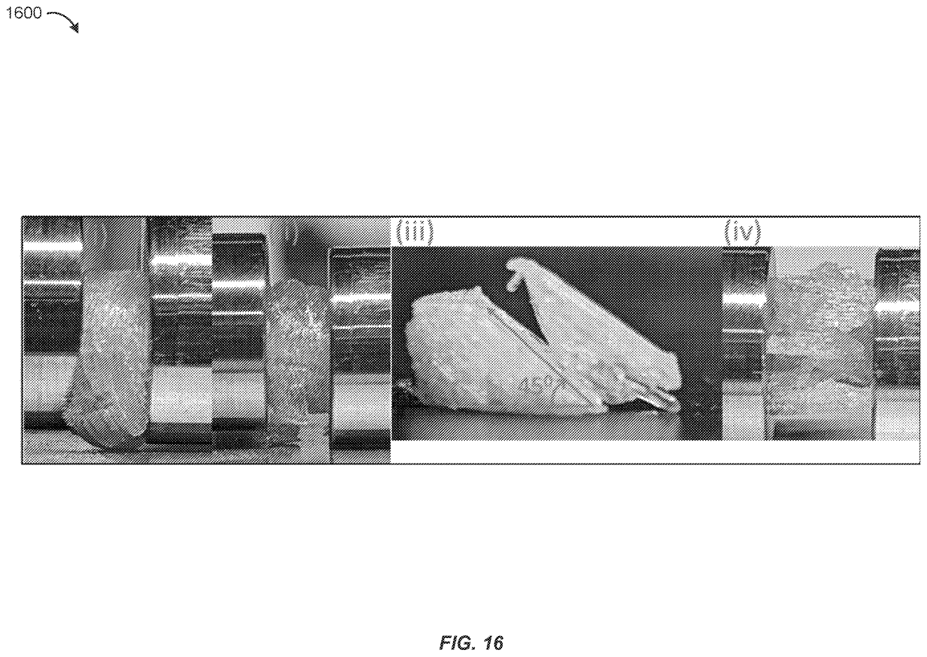

FIG. 16 shows images of printed products after breaking force test according to some examples. Image (i) shows printed product fabricated by continuous printing method and tested with vertical testing setup. Image (ii) shows printed product fabricated by batch printing method and tested with vertical testing setup. Image (iii) shows printed product fabricated by continuous printing method and tested with horizontal testing setup. Image (iv) shows printed product fabricated by batch printing method and tested with horizontal testing setup.

The printed products fabricated using the continuous method tend to break along a print split into multiple pieces. The print split may be along the axis of printing (e.g., 45°). The breaking along the print split may be attributed to how the applied force is resolved. For example, when a force is applied at a 45° angle, the applied force may be resolved into two components. One component may act along the direction along which the force is applied. The other component may act along the axis of the printed angle. In some examples, such split of the applied force prevents the crushing of the printed product and thus creates the splitting of printed product into multiple pieces along the printed axis. The splitting along printed product can be distinguished from printed product fabricated by conventional FDM printing methods (e.g., batch printing method). Printed products fabricated by conventional FDM printing methods tend to be crushed into small pieces or fragments without systematic splitting location, plane, or direction.

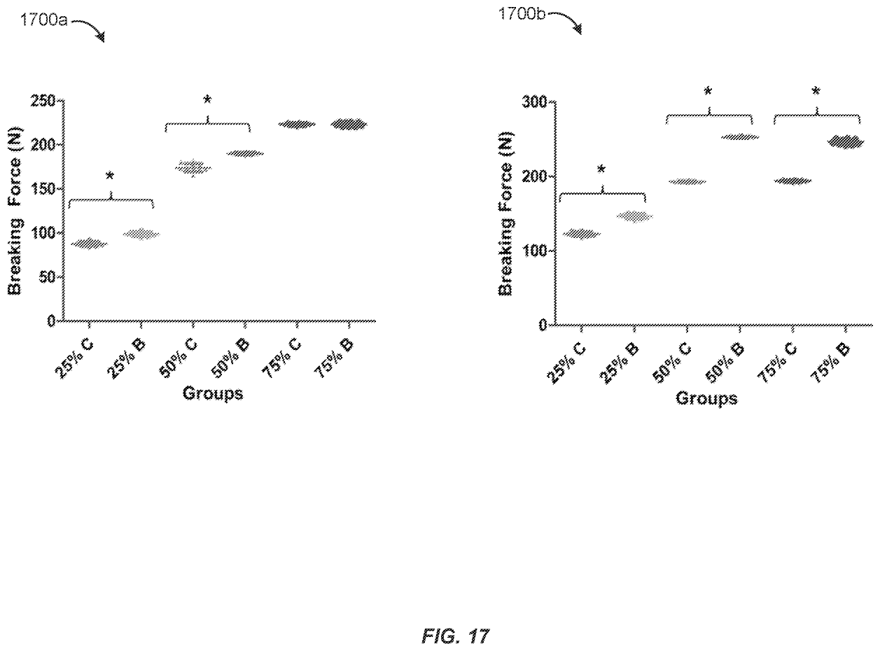

FIG. 17 shows breaking force test results of printed products according to some examples. Plot 1700a shows breaking force test results under horizontal testing setup for printed products fabricated by continuous printing method (C) and batch printing method (B) across infill densities of 25%, 50%, and 75%. Plot 1700b shows breaking force test results under vertical testing setup for printed products fabricated by continuous printing method (C) and batch printing method (B) across infill densities of 25%, 50%, and 75%. The data are represented by mean+/−standard deviation for n=10 samples. The significance of the difference is *p<0.005. As illustrated by FIG. 17, as the infill density increases, the force required to break the corresponding printed product increases. The correlation may be due to the reduced void space in printed products having higher infill density.

For printed products tested under horizontal testing setup, the breaking force tested for each printed product is different across the infill densities. In some examples (e.g., printed products having infill densities of 50% and 75%), the force required to break a printed product with a 45° printing axis is greater than the force required to break a printed product with a 0° printing axis.

For printed products tested under vertical testing setup, the printed products collapse and/or crush without showing any breakage post removal. In some examples, the printed products tested under vertical testing setup, after the testing, have reduced heights due to applied compressive force. In some examples, after the testing, the printed products samples fabricated by continuous printing method show 4 units having breakages. The printed products samples fabricated by batch printing method show 5 units having crushing. The discrepancy between printed products fabricated by different methods may be attributed to the large void spaces and weak internal structural strength of the print at low infill densities.

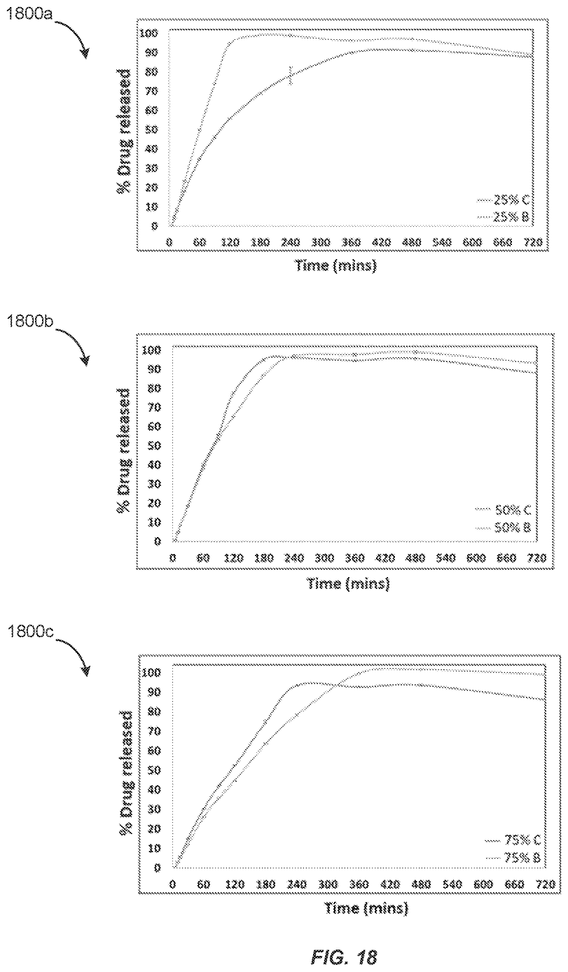

FIG. 18 shows in vitro drug release results of printed products according to some examples. Plot 1800a shows the drug release rate of printed products having a 25% infill density and fabricated by continuous printing method (C) and batch printing method (B). Plot 1800b shows the drug release rate of printed products having a 50% infill density and fabricated by continuous printing method (C) and batch printing method (B). Plot 1800c shows the drug release rate of printed products having a 75% infill density and fabricated by continuous printing method (C) and batch printing method (B). The data of plots 1800a, 1800b, and 1800c are represented by mean±standard deviation for n=3 samples.

In some examples, drug release works by first forming a thin gel layer around the tablet surface which is then followed by complete solubilization of the matrix to achieve a complete drug release. In the case of HPMC AS LG, this gel layer formation does not play a significant role as compared to the MG and HG grades which have a higher percentage of acetyl groups. The release mechanism may be thoroughly driven by swelling, solubilization, and breakdown of the polymeric matrix (e.g., HPMC AS LG) to release the entirety of its content. In some examples, the release rate of a printed product (e.g., a product including HPMC AS LG) may be governed by the solubilization or hydration of the polymeric matrix, the infill density and/or the printing orientation.

In some examples, to analyze the in vitro drug release rate of printed products, 500 mL of phosphate buffer (0.1 M, pH 6.8) may be added to dissolution vessels. The media may be maintained at 37±0.5° C. and stirred at 75 rpm. An autosampler may be used to withdraw 1 mL of the media at predetermined time points, which was then replaced with a fresh phosphate buffer. The samples may be filtered (10 μm polyethylene dissolution filters). The collected samples may be diluted two-fold with acetonitrile (HPLC grade) and the API amount may be estimated using the described method of analysis. The study may be carried out in triplicates (n=3) for all batches.

The drug release profiles of the samples may be compared using a model-independent difference factor (f₁) and similarity factor (f₂), where f₁ calculates the percent (%) difference between two curves at each time point and is a measurement of relative error between the two curves and f₂ measures the comparison of percent (%) dissolution among two curves and is the Log reciprocal square root conversion of the sum-of-squared-error.

The difference factor (f1) was calculated using the following equation:

$$f 1 = \{ \frac{[ {\sum }_{t = 1}^{n} \vert {R}_{t} - {T}_{t} \vert ]}{[ {\sum }_{t = 1}^{n} {R}_{t} ]} \} \cdot 100 \qquad ( 1 )$$

The similarity factor was calculated using the following equation:

$${f}_{2} = 50 \cdot log \{ {[ 1 + ( \frac{1}{n} ) {\sum }_{t = 1}^{n} {( {R}_{t} - {T}_{t} )}^{2} ]}^{- \frac{1}{2}} \cdot 100 \} \qquad ( 2 )$$

For equation (1) and (2), n is the number of time points, Rₜ is the percent drug release of the reference sample (batch printing process) at time point t, and Tₜ is the percent drug release of the test sample (continuous printing process) at time point t. A difference factor (f₁) close to zero (≤15) indicates minimal differences between the curves and a similarity factor (f₂) close to 100 (≥50) indicates closeness between the values of the test and reference samples.

In some examples, HPMC AS used as polymeric matrix may have a high number of acetyl and succinyl substitutions, which creates a pH threshold for solubilization. In the case of the LG grade, the pH threshold is the lowest as compared to other grades (e.g., MG and HG, pH>5.5). In some examples, the release media may be phosphate buffer (pH=6.8). A complete release for all test batches was observed in the first few hours of the study. The infill density has a major impact on the drug release from the printed product. In some examples, printed products may have a higher infill density may have a slower release profile irrespective of their release mechanism.

As illustrated by plot 1800a, for printed products having a 25% infill density but fabricated by continuous printing method (C) and batch printing method (B), their release rates are visibly different. For examples, the printed products fabricated by batch printing method (25B) show a faster release rate than the printed product fabricated by continuous printing method (25° C.). The difference in release rate may be attributed to different layer orientations of the printed products. The layer orientation may change the exposed surface area of the printed products to the neighboring release media. As illustrated by plots 1800b and 1800c, the top and bottom surfaces of a printed product fabricated by continuous printing method having a printing axis and layer orientation of 45° are completely covered and tightly packed, which leaves no void opening for the media to penetrate through or interact with the matrix. The packing remains irrespective of infill density and is a characteristic dependent on layer orientation and printing axis. In some examples, the printed products may be fabricated in a batch fashion with a 0° layer orientation. The internal pores of the printed products may be open and exposed to the surrounding media in contact with the printed product as soon as the printed products are introduced into the media. The contact can be avoided, if the top and bottom surfaces are covered with a surface layer.

As stated above, a difference factor (f₁) close to zero (≤15) indicates minimal differences between the curves and a similarity factor (f₂) close to 100 (≥50) indicates closeness between the values of the test and reference samples. Calculating these values to compare release rates from 25C and 25B samples or batch samples, a f₁ of 20.86 and a f₂ of 36.95 are obtained. The values of f₁ and f₂ attests to the fact that the release rate curves are different, and the release rate is impacted by the orientation at a 25% infill density. The different factor (f₁) and similarity factor (f₂) of printed products (n=3) fabricated using continuous printing process (C) and batch printing process (B) are summarized in Table 11.

TABLE 11

| | | Difference Factor | Similarity Factor |
|---|---|---|---|
| Reference | Test | (f₁) | (f₂) |
| 25B | 25C | 20.8620 | 36.9816 |
| 50B | 50C | 5.8145 | 65.5170 |
| 75B | 75C | 13.4370 | 54.8460 |

### REFERENCES

U.S. Pat. Nos. 10,201,503, 10,363,220, 10,624,857, 10,973,767, 11,278,499, 11,292,193, 11,364,674, 11,383,439, and 11,458,684.
U.S. Patent Application Publication No. US 2021/0154910.
Basit A W, Gaisford S, editors. 3D Printing of Pharmaceuticals. Springer International Publishing 2018. doi: 10.1007/978-3-319-90755-0.
Bruschi, editor). Mathematical Models of Drug Release. In Strategies to Modify the Drug Release from Pharmaceutical Systems; Elsevier, 2015; pp. 63-86; doi: 10.1016/B978-0-08-100092-2.00005-9.
Butreddy, A. Hydroxypropyl Methylcellulose Acetate Succinate as an Exceptional Polymer for Amorphous Solid Dispersion Formulations: A Review from Bench to Clinic. European Journal of Pharmaceutics and Biopharmaceutics 2022, 177, 289-307.
Chakka, J.; Shanthi Chede 3D Printing of Pharmaceuticals for Disease Treatment. Front Med Technol 2022.
Conradie D, “3D Printer Automated Bed Swapping System Loads From A Magazine,” Hackaday article 489626, hackaday.com/?p=489626, Aug. 3, 2021.
Crişan, A. G.; Iurian, S.; Porfire, A.; Rus, L. M.; Bogdan, C.; Casian, T.; Lucacel, R. C.; Turza, A.; Porav, S.; Tomuţă, I. QbD Guided Development of Immediate Release FDM-3D Printed Tablets with Customizable API Doses. Int J Pharm 2022, 613, doi: 10.1016/j.ijpharm.2021.121411.
Davis, D. A.; Miller, D. A.; Su, Y.; Williams, R. O. Thermally Conductive Excipient Expands KinetiSol® Processing Capabilities. AAPS PharmSciTech 2020, 21, doi: 10.1208/s12249-020-01817-7.
Dedroog, S.; Pas, T.; Vergauwen, B.; Huygens, C.; van den Mooter, G. Solid-State Analysis of Amorphous Solid Dispersions: Why DSC and XRPD May Not Be Regarded as Stand-Alone Techniques. J Pharm Biomed Anal 2020, 178, doi: 10.1016/j.jpba.2019.112937.
Duceac, I. A.; Coseri, S. Biopolymers and Their Derivatives: Key Components of Advanced Biomedical Technologies. Biotechnol Adv 2022, 61.
Dumpa, N.; Butreddy, A.; Wang, H.; Komanduri, N.; Bandari, S.; Repka, M. A. 3D Printing in Personalized Drug Delivery: An Overview of Hot-Melt Extrusion-Based Fused Deposition Modeling. Int J Pharm 2021, 600.
Fu, Y.; Kao, W. J. Drug Release Kinetics and Transport Mechanisms of Non-Degradable and Degradable Polymeric Delivery Systems. Expert Opin Drug Deliv 2010, 7, 429-444.
Hausner, D. B.; Moore, C. M. V. Regulatory Considerations for Continuous Solid Oral Dose Pharmaceutical Manufacturing. In How to Design and Implement Powder-to-Tablet Continuous Manufacturing Systems; Elsevier, 2022; pp. 301-318 ISBN 9780128134795.
Kallakunta, V.; et al. Stable Amorphous Solid Dispersions of Fenofibrate Using Hot Melt Extrusion Technology: Effect of Formulation and Process Parameters for a Low Glass Transition Temperature Drug HHS Public Access. J Drug Deliv Sci Technol 2020, 58, doi: 10.1016/jjddst.2019.101395.
Karmarkar, A.; Karmarkar, A. B. Effect of Ceolus KG-802 on the Dissolution Rate of Fenofibrate Liquisolid Tablets: Preformulation and Formulation Development Studies. Effect of Ceolus KG-802 on the Dissolution Rate of Fenofibrate Liquisolid Tablets: Preformulation and Formulation Development Studies; 2010; Vol. 4.
Kamio, T.; Onda, T. Fused Deposition Modeling 3D Printing in Oral and Maxillofacial Surgery: Problems and Solutions. Cureus 2022, doi: 10.7759/cureus.28906.
Khaled, S. A.; Burley, J. C.; Alexander, M. R.; Yang, J.; Roberts, C. J. 3D Printing of Tablets Containing Multiple Drugs with Defined Release Profiles. Int J Pharm 2015, 494, 643-650, doi: 10.1016/j.ijpharm.2015.07.067.
Klar, F.; Urbanetz, N. A. Solubility Parameters of Hypromellose Acetate Succinate and Plasticization in Dry Coating Procedures. Drug Dev Ind Pharm 2016, 42, 1621-1635, doi: 10.3109/03639045.2016.1160106.
Krueger, L.; Miles, J. A.; Popat, A. 3D Printing Hybrid Materials Using Fused Deposition Modelling for Solid Oral Dosage Forms. Journal of Controlled Release 2022, 351, 444-455.
Kukkonen, J.; Ervasti, T.; Laitinen, R. Production and Characterization of Glibenclamide Incorporated PLA Filaments for 3D Printing by Fused Deposition Modeling. J Drug Deliv Sci Technol 2022, 77, doi: 10.1016/j.jddst.2022.103843.
Kulkarni, V. R.; Kazi, M.; Shahba, A. A. W.; Radhanpuri, A.; Maniruzzaman, M. Three-Dimensional Printing of a Container Tablet: A New Paradigm for Multi-Drug-Containing Bioactive Self-Nanoemulsifying Drug-Delivery Systems (Bio-SNEDDSs). Pharmaceutics 2022, 14, doi: 10.3390/pharmaceutics14051082.
Lakkala, P.; Munnangi, S. R.; Bandari, S.; Repka, M. Additive Manufacturing Technologies with Emphasis on Stereolithography 3D Printing in Pharmaceutical and Medical Applications: A Review. Int J Pharm X 2023, 5, doi: 10.1016/j.ijpx.2023.100159.
Lu, A.; Zhang, J.; Jiang, J.; Zhang, Y.; Giri, B. R.; Kulkarni, V. R.; Aghda, N. H.; Wang, J.; Maniruzzaman, M. Novel 3D Printed Modular Tablets Containing Multiple Anti-Viral Drugs: A Case of High Precision Drop-on-Demand Drug Deposition. Pharm Res 2022, doi: 10.1007/s11095-022-03378-9.
Ma, X.; Williams, R. O. Characterization of Amorphous Solid Dispersions: An Update. J Drug Deliv Sci Technol 2019, 50, 113-124.
Meng, F.; Trivino, A.; Prasad, D.; Chauhan, H. Investigation and Correlation of Drug Polymer Miscibility and Molecular Interactions by Various Approaches for the Preparation of Amorphous Solid Dispersions. European Journal of Pharmaceutical Sciences 2015, 71, 12-24, doi: 10.1016/j.ejps.2015.02.003.
Mohapatra, S.; Kar, R. K.; Biswal, P. K.; Bindhani, S. Approaches of 3D Printing in Current Drug Delivery. Sensors International 2022, 3.
Niederquell, A.; Dujovny, G.; Probst, S. E.; Kuentz, M. A Relative Permittivity Approach for Fast Drug Solubility Screening of Solvents and Excipients in Lipid-Based Delivery. J Pharm Sci 2019, 108, 3457-3460, doi: 10.1016/j.xphs.2019.06.014.
Ong, J. J.; Castro, B. M.; Gaisford, S.; Cabalar, P.; Basit, A. W.; Pérez, G.; Goyanes, A. Accelerating 3D Printing of Pharmaceutical Products Using Machine Learning. Int J Pharm X 2022, 4, doi: 10.1016/j.ijpx.2022.100120.
Pawar, R.; Pawar, A. 3D Printing of Pharmaceuticals: Approach from Bench Scale to Commercial Development. Futur J Pharm Sci 2022, 8, 48, doi: 10.1186/s43094-022-00439-z.
Pulla, G. G.; Jagadish, G.; Lakshmi Aishwarya, K. Solid Dispersions of Fenofibrate: Comparison of Natural and Synthetic Carriers. ˜42˜The Pharma Innovation Journal 2019, 8, 42-50.
Que, C.; Lou, X.; Zemlyanov, D. Y.; Mo, H.; Indulkar, A. S.; Gao, Y.; Zhang, G. G. Z.; Taylor, L. S. Insights into the Dissolution Behavior of Ledipasvir-Copovidone Amorphous Solid Dispersions: Role of Drug Loading and Intermolecular Interactions. Mol Pharm 2019, 16, 5054-5067, doi: 10.1021/acs.molpharmaceut.9b01025.
Salazar-Gamarra, R.; Contreras-Pulache, H.; Cruz-Gonzales, G.; Binasco, S.; Cruz-Gonzales, W.; Moya-Salazar, J. Three-Dimensional Printing and Digital Flow in Human Medicine: A Review and State-of-the-Art. Applied System Innovation 2022, 5, 126, doi: 10.3390/asi5060126.
Samiei, N. Recent Trends on Applications of 3D Printing Technology on the Design and Manufacture of Pharmaceutical Oral Formulation: A Mini Review. Beni Suef Univ J Basic Appl Sci 2020, 9.
Sammaiah, P.; Chaitanya Krishna, D.; Sai Mounika, S.; Rajasri Reddy, I.; Karthik, T. Effect of the Support Structure on Flexural Properties of Fabricated Part at Different Parameters in the Fdm Process. In Proceedings of the IOP Conference Series: Materials Science and Engineering; IOP Publishing Ltd, Dec. 4, 2020; Vol. 981.
Siddharatha Dhoppalapudi; Narmada Illa A Review of Hot Melt Extrusion Paired Fused Deposition Modeling Three-Dimensional Printing for Developing Patient Centric Dosage Forms. GSC Biological and Pharmaceutical Sciences 2022, 21, 065-079, doi: 10.30574/gscbps.2022.21.2.0428.
Tambe, S.; Jain, D.; Meruva, S. K.; Rongala, G.; Juluri, A.; Nihalani, G.; Mamidi, H. K.; Nukala, P. K.; Bolla, P. K. Recent Advances in Amorphous Solid Dispersions: Preformulation, Formulation Strategies, Technological Advancements and Characterization. Pharmaceutics 2022, 14.
Thakkar, R.; Pillai, A. R.; Zhang, J.; Zhang, Y.; Kulkarni, V.; Maniruzzaman, M. Novel On-Demand 3-Dimensional (3-d) Printed Tablets Using Fill Density as an Effective Release-Controlling Tool. Polymers (Basel) 2020, 12, doi: 10.3390/POLYM12091872.
Thakkar, R.; Thakkar, R.; Pillai, A.; Ashour, E. A.; Repka, M. A. Systematic Screening of Pharmaceutical Polymers for Hot Melt Extrusion Processing: A Comprehensive Review. Int J Pharm 2020, 576.
Tracy, T.; Wu, L.; Liu, X.; Cheng, S.; Li, X. 3D Printing: Innovative Solutions for Patients and Pharmaceutical Industry. Int J Pharm 2023, 631, doi: 10.1016/j.ijpharm.2022.122480.
Varghese, R.; Salvi, S.; Sood, P.; Karsiya, J.; Kumar, D. Recent Advancements in Additive Manufacturing Techniques Employed in the Pharmaceutical Industry: A Bird's Eye View. Annals of 3D Printed Medicine 2022, 8, 100081, doi: 10.1016/j.stlm.2022.100081.
Vaz, V. M.; Kumar, L. 3D Printing as a Promising Tool in Personalized Medicine. AAPS PharmSciTech 2021, 22.
Velez, N. L.; Drennen, J. K.; Anderson, C. A. Challenges, Opportunities and Recent Advances in near Infrared Spectroscopy Applications for Monitoring Blend Uniformity in the Continuous Manufacturing of Solid Oral Dosage Forms. Int J Pharm 2022, 615.
Wallis, M.; Al-Dulimi, Z.; Tan, D. K.; Maniruzzaman, M.; Nokhodchi, A. 3D Printing for Enhanced Drug Delivery: Current State-of-the-Art and Challenges. Drug Dev Ind Pharm 2020, 46, 1385-1401.
Wang, J.; Zhang, Y.; Aghda, N. H.; Pillai, A. R.; Thakkar, R.; Nokhodchi, A.; Maniruzzaman, M. Emerging 3D Printing Technologies for Drug Delivery Devices: Current Status and Future Perspective. Adv Drug Deliv Rev 2021, 174, 294-316.
Wójcik-Pastuszka, D.; Krzak, J.; Prządka, P.; Twarda, M.; Osiński, B.; Musiał, W. Release of Bupivacaine from Artificial Ligament Implants Modified with the Silica Coating. Ceram Int 2023, 49, 2852-2859, doi: 10.1016/j.ceramint.2022.09.267.
Wu, C.; Dai, C.; Fang, G.; Liu, Y. J.; Wang, C. C. L. RoboFDM: A Robotic System for Support-Free Fabrication Using FDM. In Proceedings of the Proceedings—IEEE International Conference on Robotics and Automation; Institute of Electrical and Electronics Engineers Inc., Jul. 21, 2017; pp. 1175-1180.
Xu, W.; Sun, Y.; Dong, X.; Li, S.; Wang, H.; Xue, J.; Zheng, X. Local Order and Vibrational Coupling of the C═O Stretching Mode of γ-Caprolactone in Liquid Binary Mixtures. Sci Rep 2017, 7, doi: 10.1038/s41598-017-12030-1.
Zhang, J.; Thakkar, R.; Kulkarni, V. R.; Zhang, Y.; Lu, A.; Maniruzzaman, M. Investigation of the Fused Deposition Modeling Additive Manufacturing I: Influence of Process Temperature on the Quality and Crystallinity of the Dosage Forms. AAPS PharmSciTech 2021, 22, doi: 10.1208/s12249-021-02094-8.
Zhang, P.; Xu, P.; Chung, S.; Bandari, S.; Repka, M. A. Fabrication of Bilayer Tablets Using Hot Melt Extrusion-Based Dual-Nozzle Fused Deposition Modeling 3D Printing. Int J Pharm 2022, 624, doi: 10.1016/j.ijpharm.2022.121972.
Zheng et al., Melt extrusion deposition (MED™) 3D printing technology—A paradigm shift in design and development of modified release drug products, International Journal of Pharmaceutics, Volume 602, 2021, 120639, ISSN 0378-5173.

### STATEMENTS REGARDING INCORPORATION BY REFERENCE AND VARIATIONS

All references throughout this application, for example patent documents, including issued or granted patents or equivalents and patent application publications, and non-patent literature documents or other source material are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “1, 2, 3, 1 and 2, 1 and 3, 2 and 3, or 1, 2, and 3”.

Every formulation or combination of components described or exemplified can be used to practice the invention, unless otherwise stated. Specific names of materials are intended to be exemplary, as it is known that one of ordinary skill in the art can name the same material differently. It will be appreciated that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The invention illustratively described herein suitably may be practiced in the absence of any element, elements, limitation, or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method of manufacturing a printed product, the method comprising:
providing an initial mixture including at least one polymeric material and at least one active pharmaceutical ingredient (API);
feeding the initial mixture into a heating barrel;
heating the initial mixture to a temperature that reduces the viscosity of the at least one polymeric material;
pumping the initial mixture through a die to form a filament;
heating the filament to a fusing temperature; and
depositing the filament on a receiving surface at an oblique angle to form a printed product.

2. (canceled)

3. (canceled)

4. The method of claim 1, wherein the receiving surface is a dynamic bed that moves continuously along an axis and wherein the method further comprises transporting the printed product on the dynamic bed.

5. (canceled)

6. The method of claim 1, further comprising measuring a quality or characteristic of the printed product using at least one in-line measurement device.

7. The method of claim 6, wherein the at least one in-line measurement device includes a digital imaging unit, an in-line UV-VIS imaging unit, an infrared spectrometer, a back pressure sensor, or a NIR probe.

8. (canceled)

9. (canceled)

10. (canceled)

11. (canceled)

12. The method of claim 1, further comprising injecting a second API into the printed product using a syringe.

13. (canceled)

14. (canceled)

15. (canceled)

16. (canceled)

17. (canceled)

18. A method of manufacturing a filament, the method comprising:
providing an initial mixture including at least one polymeric material and at least one active pharmaceutical ingredient (API);
feeding the initial mixture into a heating barrel;
heating the initial mixture at a temperature that reduces the viscosity of the at least one polymeric material; and
pumping the initial mixture through a die to form a filament.

19. (canceled)

20. The method of claim 18, wherein the filament contains at least one API in semi-crystalline state or at least one API in crystalline state.

21. (canceled)

22. A printed product comprising:
at least one active pharmaceutical ingredient (API);
and at least one polymeric material, wherein the printed product comprises a fused multilayer structure with one or more layers of the fused multilayer structure comprising the at least one active pharmaceutical ingredient (API) and the at least one polymeric material.

23. The printed product of claim 22, wherein layers of the fused multilayer structure are arranged at an oblique angle to a surface of the printed product.

24. The printed product of claim 22, wherein the API is in a gel state, liquid state, a crystalline state, a semi-crystalline state or an amorphous state.

25. (canceled)

26. (canceled)

27. The printed product of claim 22, wherein the at least one API is Nifedipine, Aspirin, chloroquine diphosphate, or Ibuprofen.

28. The printed product of claim 22, wherein the at least one polymeric material is hydroxpropylmethyl cellulose, hydroxypropyl cellulose, or hydroxpropylmethyl cellulose.

29. The printed product of claim 22, further comprising at least one plasticizer or at least one excipient.

30. (canceled)

31. The printed product of claim 22, further comprising a coating surrounding at least a portion of the fused multilayer structure.

32. The printed product of claim 31, wherein the coating comprises a second fused multilayer structure surrounding at least a portion of the fused multilayer structure.

33. The printed product of claim 22, wherein the at least one API or the at least one polymeric material has a porosity greater than zero.

34. The printed product of claim 22, further comprising a semi-solid substance that is at least partially surrounded by or at least partially internal to the fused multilayer structure.

35. (canceled)

36. The printed product of claim 22, wherein the fused multilayer structure comprises a first set of fused layers including a first API and a second set of fused layers including a second API, wherein the first set of fused layers and the second set of fused layers are fused to one another; or wherein the fused multilayer structure comprises a core part and a shell part, wherein the core part includes a first set of fused layers including at least one API, wherein the shell part includes fused material surrounding the core part.

37. (canceled)

38. (canceled)

39. The printed product of claim 22, further comprising a markline in the fused multilayer structure, wherein the markline comprises material deposited on an outer surface of the fused multilayer structure or a recessed region in the fused multilayer structure.

40. The printed product of claim 22, wherein the printed product is or comprises an amorphous solid dispersion (ASD), wherein the ASD includes particles, wherein each particle comprises the API dissolved or suspended in the polymeric material, wherein the particles form or comprise an amorphous and non-crystalline structure.
